(19) United States
(12) Patent Application Publication
(10) Pub. No.: US 2026/0131145 A1
(43) Pub. Date: May 14, 2026

(54) DEVICES, SYSTEMS, AND METHODS FOR AURICULAR VAGUS NERVE STIMULATION

(71) Applicants: THE UNITED STATES GOVERNMENT AS REPRESENTED BY THE DEPARTMENT OF VETERANS AFFAIRS, Washington, DC (US); UNIVERSITY OF PITTSBURGH - OF THE COMMONWEALTH SYSTEM OF HIGHER EDUCATION, Pittsburgh, PA (US)

(72) Inventors: Michael A. Urbin, Pittsburgh, PA (US); Rory Cooper, Pittsburgh, PA (US)

(21) Appl. No.: 19/119,679

(22) PCT Filed: Oct. 13, 2023
(86) PCT No.: PCT/US2023/076797

§ 371 Date: Apr. 9, 2025

Related U.S. Application Data
(60) Provisional application No. 63/416,225, filed on Oct. 14, 2022.

(51) Int. Cl. A61N 1/36; A61N 1/04

(52) U.S. Cl. CPC A61N 1/36036; A61N 1/0456; A61N 1/0472

(57) ABSTRACT

Disclosed is apparatus for providing stimulation to an ear of a user. The apparatus has a body that is at least partly receivable into the ear canal. A plurality of electrodes are coupled to the body. The plurality of electrodes are positioned to contact surfaces of the ear. Methods and systems for applying one or more electrical pulses to an ear are also disclosed.

### CROSS-REFERENCE TO RELATED APPLICATION

This application claims priority to and the benefit of the filing date of U.S. Provisional Patent Application No. 63/416,225, filed Oct. 14, 2022, the entirety of which is hereby incorporated by reference herein.

### BACKGROUND

Treatment of symptoms associated with various disease states, such as certain psychiatric disorders, neurological disorders, chronic inflammatory disorders, or other disorders described further herein can be inadequate. Moreover, conventional noninvasive treatments for such diseases are even more limited in availability or efficacy. Accordingly, a safe and effective treatment is desirable. Moreover, noninvasive methods to enhance learning across a variety of performance contexts are desirable.

### BRIEF SUMMARY

Implantable vagus nerve stimulation (VNS) is FDA-approved for treatment of depression and epilepsy and is also under investigation for treatment of symptoms associated with a broad array of clinical disorders. VNS devices require implantation via medical procedures that are invasive and costly, with potential for adverse side effects. Evidence of innervation by the auricular branch of the vagus nerve in the external ear dermatome has led to the idea that the vagus nerve can be recruited noninvasively with transcutaneous stimulation. Although some studies in animals and humans have shown promising results with regard to the therapeutic potential of transcutaneous auricular vagus nerve stimulation (taVNS), findings in the existing scientific literature are mixed, which is due, at least in part, to anatomical variation and sparse biomarker evidence/confirmation. Current barriers to progress include a lack of knowledge regarding which locations to target and which stimulation parameters to apply in order to engage relevant neural structures and elicit therapeutic effects. The embodiments described herein address the complexity and individual variation of cutaneous innervation and external ear anatomy.

Disclosed are methods of activating (optionally, acutely activating) one or more neuromodulatory systems (e.g., optionally, the noradrenergic system) in a subject comprising applying one or more electrical pulses to an ear of the subject, wherein the one or more electrical pulses are sufficient to activate (e.g., directly or secondarily) the one or more neuromodulatory systems (e.g., optionally, the noradrenergic system) in the subject.

Disclosed are methods of treating a disease or disorder in a subject comprising applying one or more electrical pulses to an ear of the subject, wherein the one or more electrical pulses are sufficient to activate (e.g., directly or secondarily) one or more neuromodulatory systems (e.g., optionally, the noradrenergic system) in the subject such that the disease or disorder in the subject is treated.

Disclosed are methods of treating a subject in need thereof comprising applying one or more electrical pulses to an ear of the subject, wherein the one or more electrical pulses are sufficient to activate (e.g., directly or secondarily) the one or more neuromodulatory systems (e.g., optionally, the noradrenergic system) of the subject in need thereof.

Disclosed are methods to support learning in a subject in need thereof comprising applying one or more electrical pulses to an ear of the subject, wherein the one or more electrical pulses are sufficient to activate (e.g., directly or secondarily) the one or more neuromodulatory systems (e.g., optionally, the noradrenergic system) of the subject in need thereof, wherein the subject in need thereof has enhanced learning.

Disclosed is apparatus for use with a user having an ear, the ear having an ear canal, a tragus, and a concha. The apparatus comprises a body that is at least partly receivable into the ear canal, the body having an outer surface. A plurality of electrodes are coupled to the body. The plurality of electrodes are positioned to contact surfaces of the ear.

A system can comprise the apparatus and a stimulation generator in communication with the first electrical conductor and the second electrical conductor. The stimulation generator can be configured to generate current for providing electrical stimulation through or between the first and second electrodes of the apparatus.

Additional advantages of the disclosed method and compositions will be set forth in part in the description which follows, and in part will be understood from the description, or may be learned by practice of the disclosed method and compositions. The advantages of the disclosed method and compositions will be realized and attained by means of the elements and combinations particularly pointed out in the appended claims. It is to be understood that both the foregoing general description and the following detailed description are exemplary and explanatory only and are not restrictive of the invention as claimed.

### BRIEF DESCRIPTION OF THE DRAWINGS

The accompanying drawings, which are incorporated in and constitute a part of this specification, illustrate several embodiments of the disclosed method and compositions and together with the description, serve to explain the principles of the disclosed method and compositions.

FIG. 1A and FIG. 1B show an example of the effects of vagus nerve stimulation (VNS) during extinction training. FIG. 1A shows the results from auditory fear conditioning of groups undergoing extinction training paired with sham stimulation or VNS or extended extinction. Note that extinction training paired with VNS accelerates extinction of the fear response. FIG. 1B shows the results from a PTSD model during extinction training paired with either sham stimulation or VNS. Note that the VNS group shows enhanced extinction of the fear response.

FIG. 2A and FIG. 2B show an exemplary motor training paradigm used in rodent studies to evaluate the effects of VNS to enhance rehabilitation outcomes after damage to the corticospinal tract.

FIG. 3A and FIG. 3B show an example of the effects of VNS during motor retraining after stroke. Both FIG. 3A shows the number of successful attempts at achieving target force and FIG. 3B shows the peak force achieved is greatest in the group that trained with VNS that temporally coincides with successful performance.

FIG. 4 shows afferent innervation from the auricular branch of the vagus nerve in the external ear.

FIG. 5A and FIG. 5B are eye tracking studies. FIG. 5A shows timing of events during an eye tracking paradigm (left) with 25 Hz pulse trains applied at each multiplier of perceptual threshold (right). FIG. 5B shows representative recording of pupil diameter during eye tracking procedures. A pupillary response is shown with the various response features (i.e., size and timing) annotated. Gray vertical bar corresponds to the stimulation epoch.

FIG. 6A and FIG. 6B are studies on pupil diameter. FIG. 6A shows the acceleration-time profile of pupil diameter in two representative subjects when 25 Hz (gray) and 300 Hz (black) pulse frequencies are administered. FIG. 6B shows corresponding pupil diameter at each pulse frequency. Circles indicate the time of peak positive acceleration, and the lighter shade vertical bar corresponds to the stimulation epoch. Note that the greatest rate of change in pupil diameter occurs at the time of peak acceleration.

FIG. 7A and FIG. 7B are studies on pupil diameter. FIG. 7A shows the change in pupil diameter by location and pulse amplitude in a sample (n=19) of neurologically-intact humans (*p<0.05). Note the gradation by pulse amplitudes at and above perceptual threshold, particularly for the canal location. Error bars represent standard error of the mean. FIG. 7B shows waveform-averaged pupil diameter recordings from a representative subject when pulse trains were applied to different landmarks [i.e., canal, concha, and lobe] with pulse amplitudes at and above PT [i.e., 1.0xPT (left), 1.5xPT (middle), and 2.0xPT (right)]. Triangles and circles correspond to the time of peak positive acceleration and peak dilation, respectively. Gray vertical bar corresponds to the stimulation epoch.

FIG. 8A and FIG. 8B are studies on pupil dilation. FIG. 8A shows area under the curve (AuC) of the pupillary response between the times of peak positive acceleration and peak dilation by location. AuC modulated with pulse frequency when pulse trains were applied to the canal location. 300 Hz pulse frequency produced pupillary responses with greater AuC when pulse trains were applied to the canal versus the concha and lobe (*p<0.05). FIG. 8B shows latencies of peak acceleration and dilation by pulse frequency. Higher pulse frequency reduced the latency of both events (*p<0.05). Error bars represent standard error of the mean.

FIG. 9A and FIG. 9B show pupil diameter. FIG. 9A shows pupil diameter recordings while single, 25 Hz pulse trains were applied to the canal location with pulse amplitudes at and above perceptual threshold (i.e., 1.0xPT-2.0xPT, left to right). FIG. 9B shows pupil diameter recordings while single, 300 Hz pulse trains were applied to the canal location with pulse amplitudes at and above perceptual threshold (1.0xPT-2.0xPT, left to right). Thin-solid and broken traces correspond to instances where single pulse trains did or did not elicit pupillary responses, respectively. The bold trace corresponds to the waveform-averaged pupil diameter recording in the 0.0xPT amplitude condition that was randomized into each block of trials. Note that single pulse trains modulate the noradrenergic biomarker within the stimulation epoch, providing evidence of activation. Triangles correspond to the time of peak positive acceleration (inverted if pupillary response is not elicited), and circles correspond to peak dilation. Error bars represent standard error of the mean.

FIG. 10 is a perspective view of an apparatus for providing stimulation in accordance with embodiments disclosed herein, shown as partially transparent.

FIG. 11 is another perspective view of the apparatus of FIG. 10, shown as opaque.

FIG. 12 is another perspective view of the apparatus of FIG. 10.

FIG. 13 is another perspective view of the apparatus of FIG. 10.

FIG. 14A shows a portion of an external ear and the apparatus of FIG. 10 positioned therein, with the apparatus shown as opaque. FIG. 14B shows the portion of an external ear and the apparatus of FIG. 10 positioned therein, with the apparatus shown as partially transparent.

FIG. 15A shows a perspective view of the apparatus of FIG. 10 as opaque. FIG. 15A shows a perspective view of the apparatus of FIG. 10 as partially transparent.

FIG. 16A shows an anterior view of a portion of the ear and the apparatus of FIG. 10 positioned therein, with the apparatus shown as opaque. FIG. 16B shows the anterior view of a portion of the ear and the apparatus of FIG. 10 positioned therein, with the apparatus shown as partially transparent.

FIG. 17A shows a perspective view of the apparatus of FIG. 10 as opaque. FIG. 17A shows a perspective view of the apparatus of FIG. 10 as partially transparent.

FIG. 18A shows a posterior view of a portion of the ear and the apparatus of FIG. 10 positioned therein, with the apparatus shown as opaque. FIG. 18B shows the posterior view of a portion of the ear and the apparatus of FIG. 10 positioned therein, with the apparatus shown as partially transparent.

FIG. 19 is a system for providing stimulation as disclosed herein.

FIG. 20 is an exemplary computing device of the system of FIG. 19.

FIG. 21 is a rear view of an exemplary apparatus for providing stimulation in accordance with embodiments disclosed herein.

FIG. 22 is a front view of the exemplary apparatus of FIG. 21.

FIGS. 23-28 show perspective views of different exemplary apparatuses, collectively illustrating variations in geometry to match the shapes of the ears of different subjects.

FIG. 31A shows an assembly for providing stimulation as disclosed herein. FIG. 31B shows a partial close-up view of the apparatus for providing stimulation.

FIG. 32A shows an assembly for providing stimulation as disclosed herein. FIG. 32B shows a partial close-up view of the apparatus for providing stimulation.

### DETAILED DESCRIPTION

The disclosed method and compositions may be understood more readily by reference to the following detailed description of particular embodiments and the Example included therein and to the Figures and their previous and following description.

It is to be understood that the disclosed method and compositions are not limited to specific synthetic methods, specific analytical techniques, or to particular reagents unless otherwise specified, and, as such, may vary. It is also to be understood that the terminology used herein is for the purpose of describing particular embodiments only and is not intended to be limiting.

Disclosed are materials, compositions, and components that can be used for, can be used in conjunction with, can be used in preparation for, or are products of the disclosed method and compositions. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. Thus, if a class of molecules A, B, and C are disclosed as well as a class of molecules D, E, and F and an example of a combination molecule, A-D is disclosed, then even if each is not individually recited, each is individually and collectively contemplated. Thus, is this example, each of the combinations A-E, A-F, B-D, B-E, B-F, C-D, C-E, and C-F are specifically contemplated and should be considered disclosed from disclosure of A, B, and C; D, E, and F; and the example combination A-D. Likewise, any subset or combination of these is also specifically contemplated and disclosed. Thus, for example, the sub-group of A-E, B-F, and CE are specifically contemplated and should be considered disclosed from disclosure of A, B, and C; D, E, and F; and the example combination A-D. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods, and that each such combination is specifically contemplated and should be considered disclosed.

### A. Definitions

It is understood that the disclosed method and compositions are not limited to the particular methodology, protocols, and reagents described as these may vary. It is also to be understood that the terminology used herein is for the purpose of describing particular embodiments only, and is not intended to limit the scope of the present invention which will be limited only by the appended claims.

It must be noted that as used herein and in the appended claims, the singular forms “a,” “an,” and “the” can include both single and plural references unless the context clearly dictates otherwise. Thus, for example, unless the context dictates otherwise, reference to “an electrical pulse” includes aspects in which only one electrical pulse is provided, as well as aspects in which a plurality of such electrical pulses are provided.

The term “auricular vagus nerve stimulation” (e.g., transcutaneous auricular vagus nerve stimulation) refers to activating neuromodulatory systems directly or secondarily through stimulation applied to the external ear. Other neurotransmitter systems (e.g., cholinergic, serotonergic, dopaminergic) may be primarily or secondarily activated by noninvasive and invasive vagus nerve stimulation.

As used herein, the term “subject,” “patient,” or “individual” can be used interchangeably and refer to any organism to which an electrical pulse of this invention may be applied or administered, e.g., for experimental, diagnostic, and/or therapeutic purposes. Typical subjects include animals (e.g., mammals such as non-human primates, and humans; avians; domestic household or farm animals such as cats, dogs, sheep, goats, cattle, horses and pigs; laboratory animals such as mice, rats and guinea pigs; rabbits; fish; reptiles; zoo and wild animals). Typically, “subjects” are animals, including mammals such as humans and primates; and the like. The term does not denote a particular age or sex.

By “treat” is meant to administer or apply a therapeutic, such as an electrical pulse, to a subject, such as a human or other mammal (for example, an animal model), that has a disease or disorder that can be treated by vagus nerve stimulation (e.g., invasive or noninvasive vagus nerve stimulation) or has an increased susceptibility for developing a disease or disorder that can be treated by (invasive or noninvasive) vagus nerve stimulation, in order to prevent, reduce, or delay a worsening of the effects or symptoms of the disease or disorder, to partially or fully reverse the effects or symptoms of the disease or disorder, or ameliorate a symptom of the disease or disorder (e.g. psychiatric and neurologic disorders and chronic inflammatory disorders).

By “ameliorate” is meant to lessen at least one indicator, sign, or symptom of an associated disease, disorder, or condition. The severity of indicators may be determined by subjective or objective measures, which are known to those skilled in the art.

By “prevent” is meant to minimize the chance that a subject who has an increased susceptibility for developing a disease or disorder that can be treated vagus nerve stimulation will develop the disease or disorder or symptoms associated with the disease or disorder.

The phrase “acute activation” or “acutely activating” refers to activation that occurs on millisecond timescales during the time course and/or immediately following the time of stimulation. For example, acutely activating neuromodulatory systems refers to activating neuromodulatory systems at the time of stimulation or less than 1 second thereafter. Acute activation can include direct or secondary activation.

“Optional” or “optionally” means that the subsequently described event, circumstance, or material may or may not occur or be present, and that the description includes instances where the event, circumstance, or material occurs or is present and instances where it does not occur or is not present.

Ranges may be expressed herein as from “about” one particular value, and/or to “about” another particular value. When such a range is expressed, also specifically contemplated and considered disclosed is the range from the one particular value and/or to the other particular value unless the context specifically indicates otherwise. Similarly, when values are expressed as approximations, by use of the antecedent “about,” it will be understood that the particular value forms another, specifically contemplated embodiment that should be considered disclosed unless the context specifically indicates otherwise. It will be further understood that the endpoints of each of the ranges are significant both in relation to the other endpoint, and independently of the other endpoint unless the context specifically indicates otherwise. Finally, it should be understood that all of the individual values and sub-ranges of values contained within an explicitly disclosed range are also specifically contemplated and should be considered disclosed unless the context specifically indicates otherwise. The foregoing applies regardless of whether in particular cases some or all of these embodiments are explicitly disclosed.

Optionally, in some aspects, when values or characteristics are approximated by use of the antecedents “about,” “substantially,” or “generally,” it is contemplated that values within up to 15%, up to 10%, up to 5%, or up to 1% (above or below) of the particularly stated value or characteristic can be included within the scope of those aspects.

Unless defined otherwise, all technical and scientific terms used herein have the same meanings as commonly understood by one of skill in the art to which the disclosed method and compositions belong. Although any methods and materials similar or equivalent to those described herein can be used in the practice or testing of the present method and compositions, the particularly useful methods, devices, and materials are as described. Publications cited herein and the material for which they are cited are hereby specifically incorporated by reference. Nothing herein is to be construed as an admission that the present invention is not entitled to antedate such disclosure by virtue of prior invention. No admission is made that any reference constitutes prior art. The discussion of references states what their authors assert, and applicants reserve the right to challenge the accuracy and pertinence of the cited documents. It will be clearly understood that, although a number of publications are referred to herein, such reference does not constitute an admission that any of these documents forms part of the common general knowledge in the art.

Throughout the description and claims of this specification, the word “comprise” and variations of the word, such as “comprising” and “comprises,” means “including but not limited to,” and is not intended to exclude, for example, other additives, components, integers or steps. In particular, in methods stated as comprising one or more steps or operations it is specifically contemplated that each step comprises what is listed (unless that step includes a limiting term such as “consisting of”), meaning that each step is not intended to exclude, for example, other additives, components, integers or steps that are not listed in the step.

### B. Methods of Activating One or More Neuromodulatory Systems

The vagus nerve has been a target of neuromodulation technologies that aim to treat symptoms associated with a wide array of disease states. Noninvasive approaches have been developed to overcome the need for surgical procedures and to avoid the cost of implantable systems. Demonstration of autonomic engagement by way of noninvasive approaches is needed to confirm that the targeted neural pathways are activated to elicit the intended effect(s). Evidence to this end is also a necessary step for therapeutic applications that require synchronized activation of relevant neural pathways with behavioral, environmental, or task-related events. It has been shown that brief pulse trains of electrical current applied to external ear locations thought to be innervated by the auricular branch of the vagus nerve drive acute, autonomic responses in the form of pupillary dilation in humans, enhancing the potential of targeted neuromodulation used to enhance learning across a variety of human performance contexts and to improve quality of life for individuals suffering from chronic health conditions.

Neuromodulatory systems, when activated, promote neural reorganization of the nervous system. Vagus nerve stimulation is thought to elicit release of chemicals in the brain, which may be engaged by recruitment of vagal afferents innervating the dermatome of the external ear.

Disclosed are methods comprising applying one or more electrical pulses to an ear of a subject, wherein the one or more electrical pulses are sufficient to activate (optionally, acutely activate) one or more neuromodulatory systems of the subject. In various aspects, the one or more neuromodulatory systems can include the noradrenergic, serotonergic, dopaminergic, and/or cholinergic systems. For example, activation of one or more of the neuromodulatory systems can include activation of any combination of the noradrenergic, serotonergic, dopaminergic, and/or cholinergic systems. In some aspects, “electrical pulse” can be used interchangeably with “electrical impulse.” In some aspects, one or more electrical pulses is the same as one or more pulses of electrical current. In some aspects, one or more electrical pulses comprise a stimulation train and several stimulation trains can be applied in the context of a single treatment or application. In some aspects, a stimulation train is a group of electrical impulses that are close together in time.

Disclosed are methods of activating (optionally, acutely activating) one or more neuromodulatory systems in a subject comprising applying one or more electrical pulses to an ear of the subject, wherein the one or more electrical pulses are sufficient to activate the one or more neuromodulatory systems of the subject.

In some aspects, activating (optionally, acutely activating) the one or more neuromodulatory systems comprises stimulating the vagus nerve. In some aspects, acutely activating the one or more neuromodulatory systems comprises an activation that can last long after stimulation has ended. In some aspects, activating (optionally, acutely activating) the one or more neuromodulatory systems comprises a stimulation that engages the target (e.g., noradrenergic mechanism) on short/millisecond time scales (e.g., less than 1 s).

In some aspects, one or more electrical pulses can be applied to the external car of a subject. In some aspects, one or more electrical pulses can be applied to the ear canal of a subject. In some aspects, the ear canal can also be referred to as the external acoustic meatus. In some aspects, one or more electrical pulses can be applied to the cymba and cavum concha of a subject. In some aspects, one or more electrical pulses can be applied to the tragus of the subject. In some aspects, stimulating the ear with one or more electrical pulses can also be referred to as transcutaneous auricular vagus nerve stimulation (ta VNS). ta VNS refers to a noninvasive stimulation that can result in activation (optionally, acute activation) of the neuromodulatory systems.

In some aspects, the one or more electrical pulses are applied unilaterally to the subject. In some aspects, the one or more electrical pulses are applied only to the external ear of the subject. In some aspects, one or more electrical pulses can be applied to the cymba and cavum concha of a subject. In some aspects, the one or more electrical pulses are applied only to the left ear of the subject. In some aspects, the one or more electrical pulses are applied only to the external ear of the left ear of the subject. In some aspects, the one or more electrical pulses are applied only to the ear canal of the left ear of the subject. In some aspects, one or more electrical pulses can be applied to the tragus of the left ear of the subject. Thus, in some aspects, one or more electrical pulses are not applied to the right ear of the subject. In some aspects, the one or more electrical pulses are applied only to the right ear of the subject. In some aspects, the one or more electrical pulses are applied only to the external ear of the right ear of the subject. In some aspects, the one or more electrical pulses are applied only to the ear canal of the right ear of the subject. In some aspects, one or more electrical pulses can be applied to the tragus of the right ear of the subject. Thus, in some aspects, one or more electrical pulses are not applied to the left ear of the subject.

In some aspects, the one or more electrical pulses are applied bilaterally to the subject. Thus, in some aspects, the one or more electrical pulses are applied to both the right and left ear of the subject.

Activation (e.g., acute activation) of the neuromodulatory systems can be important for therapeutic applications. In some aspects, activation of the neuromodulatory systems must coincide with an environmental, behavioral, or task-related event in order for the activation to provide a therapeutic effect. For example, activation can be via auricular vagus nerve stimulation that is provided or occurs at approximately the same time as an environmental, behavioral, or task-related event and can result in a therapeutic effect. Examples of an environmental, behavioral, or task-related event can be, but are not limited to, exposure to noxious stimulus that is otherwise benign, but through negative association, becomes noxious. Additional examples of behavioral events include performance of a motor or cognitive task.

In some aspects, applying one or more electrical pulses to the ear of the subject comprises positioning an object or device within the ear of the subject and generating electrical pulses with the object or device, wherein the electrical pulses are transmitted through the ear to the vagus nerve in the subject. In some aspects, the methods can further comprise positioning a return via a snap electrode on a lateral mastoid or spinous process.

The ear comprises an auricular branch of the vagus nerve. In some aspects, the auricular branch of the vagus nerve is in the external ear. The vagus nerve is comprised of afferent and efferent fibers, such that electrical current is transduced into neural signals that are, in turn, transmitted within the central nervous system (CNS). The auricular branch of the vagus nerve innervates the skin of the external auditory meatus, inner wall of the tragus, and concha cymba/cavum.

In some aspects, activation of neuromodulatory systems is confirmed by presence of pupil dilation. In some aspects, assessing pupil dilation comprises looking for a change in pupil diameter. In some aspects, activation of neuromodulatory systems can be confirmed by the change in pupil diameter, wherein the change in pupil diameter is an increase in pupil dilation. In some aspects, assessing pupil dilation comprises establishing perceptual thresholds and measuring changes in pupil dilation from baseline to after stimulation onset. In some aspects, establishing perceptual thresholds comprises determining the minimum electrical current amplitude needed to evoke a percept (i.e. conscious awareness that a stimulus is present) from a subject. In some aspects, measuring changes in pupil dilation while stimulating a given location on the external ear can be achieved using electrical current amplitudes below the established perceptual threshold, at the established perceptual threshold, and/or above the established perceptual threshold.

In some aspects, the one or more electrical pulses are administered at a frequency of between 1 Hz and 10,000 Hz. In some aspects, the one or more electrical pulses are administered at a frequency of between 100 Hz and 300 Hz, between 200 Hz and 400 Hz, between 250 Hz and 350 Hz, between 300 Hz and 400 Hz. In some aspects, the one or more electrical pulses are administered at a frequency of 100, 150, 200, 250, 300, 350, 400, 450, or 500 Hz. In some aspects, the one or more electrical pulses are administered at a frequency of 100, 1000, 2000, 3000, 4000, 5000, 6000, 7000, 8000, 9000, or 10,000 Hz. In some aspects, the one or more electrical pulses are administered at a frequency of 300 Hz.

In some aspects, applying one or more electrical pulses to an ear of the subject occurs for a duration of about 1 ms to about 1000 ms. In further aspects, the duration can be greater than 1000 ms (e.g., optionally, 1000 ms to about 5000 ms, or more). In some aspects, applying one or more electrical pulses to an car of the subject occurs for a duration of about 500 ms to about 800 ms. In some aspects, applying one or more electrical pulses to an ear of the subject occurs for a duration of about 100, 150, 200, 250, 300, 350, 400, 450, 500, 550, 600, 650, 700, 750, or 800 ms. In some aspects, applying one or more electrical pulses to an ear of the subject occurs for a duration of about 30 seconds to 30 minutes. In some aspects, applying one or more electrical pulses to an ear of the subject occurs for a duration of about 1, 2, 3, 4, 5, 6, 7, 8, 9, 10, 15, 20, 25, or 30 minutes.

In some aspects, applying one or more electrical pulses to an ear of the subject occurs with a pulse width of 100 μs and 1,000 μs. In some aspects, the one or more electrical pulses have a pulse width of between 100 μs and 300 μs, between 200 μs and 400 μs, between 250 μs and 350 μs, between 300 μs and 400 μs. In some aspects, the one or more electrical pulses have a pulse width of 100, 150, 200, 250, 300, 350, 400, 450, or 500 μs. In some aspects, the one or more electrical pulses have a pulse width of 300 μs.

In some aspects, current amplitude can be normalized on a subject by subject basis. Because the current amplitude is determined based on perceptual threshold and each subject can have a slightly varied perceptual threshold, the current amplitude is not necessarily a set value. Although the current amplitude can vary from subject to subject, in some aspects there can be a critical window since the effects of stimulation can interfere with learning, performance, or physiological functions. Thus, the current amplitude can be titrated relative to perceptual threshold. In some aspects, the amplitude can be 0-10 mA.

### C. Methods to Support Learning, Unlearning, or Treating Symptoms of Clinical Disorders

Disclosed herein are methods of supporting learning or re-learning in a subject. Disclosed herein are methods in a subject comprising applying one or more electrical pulses to an ear of the subject, wherein the one or more electrical pulses are sufficient to activate (optionally, acutely activate) one or more neuromodulatory systems (e.g., the noradrenergic system) of the subject thereby supporting learning in the subject. For example, stimulating the vagus nerve to activate the one or more neuromodulatory systems allows enhanced memory consolidation during motor learning or re-learning. For example, a subject having undergone a stroke can undergo motor rehabilitation to re-train muscles weakened by or movements impaired by the stroke. Vagus nerve stimulation, such as taVNS, can enhance the effect of the re-training. In some aspects, vagus nerve stimulation must be paired with re-trained movements and not delayed to allow activation of the one or more neuromodulatory systems to coincide with movements.

Disclosed are methods of treating a subject in need thereof comprising applying one or more electrical pulses to an ear of the subject, wherein the one or more electrical pulses are sufficient to activate the one or more neuromodulatory systems (e.g., the noradrenergic system) of the subject in need thereof. In some aspects, a subject in need thereof can be a subject who has suffered a stroke. In some aspects, a subject in need thereof can be a subject having a psychiatric disorder, neurological disorder, chronic inflammatory disorder, or other disorder. In some aspects, the psychiatric disorder can be, but is not limited to, post-traumatic stress disorder (PTSD), anxiety, depression, or substance abuse. In some aspects, the neurologic disorders can be, but are not limited to, paretic syndrome (i.e., muscle weakness, spasticity, etc.) resulting from damage to the corticospinal tract due to stroke, spinal cord injury, or traumatic brain injury. In some aspects, other neurological disorders can be, but are not limited to epilepsy, nystagmus, neuropathic pain, disorders of cognition/consciousness, or tinnitus. In some aspects, the chronic inflammatory disorders can be fibromyalgia, migraine headaches, or obesity. In some aspects, other disorders can be lung injury, cardiovascular disease/atherosclerosis, or diabetes. In some aspects, a subject in need thereof is a healthy subject. For example, any healthy subject can be treated with one or more electrical pulses in the ear to activate the one or more neuromodulatory systems (e.g., the noradrenergic system) of the subject in order to facilitate learning in the subject.

Disclosed are methods of treating a disease or disorder in a subject comprising applying one or more electrical pulses to an ear of the subject, wherein the one or more electrical pulses are sufficient to activate the one or more neuromodulatory systems (e.g., the noradrenergic system) of the subject such that the disease or disorder in the subject is treated. In some aspects, the disease or disorder can be a psychiatric disorder, neurological disorder, chronic inflammatory disorder, or other disorders. In some aspects, the psychiatric disorder can be, but is not limited to, post-traumatic stress disorder (PTSD), anxiety, depression, or substance abuse. In some aspects, the neurologic disorders can be, but are not limited to, paretic syndrome (i.e., muscle weakness, spasticity, etc.) resulting from damage to the corticospinal tract due to stroke, spinal cord injury, or traumatic brain injury. In some aspects, other neurological disorders can be but are not limited to epilepsy, nystagmus, neuropathic pain, disorders of cognition/consciousness, or tinnitus. In some aspects, the chronic inflammatory disorders can be fibromyalgia, migraine headaches, or obesity. In some aspects, other disorders can be lung injury, cardiovascular disease/atherosclerosis, or diabetes.

Disclosed are methods of treating a disease or disorder in a subject comprising applying one or more electrical pulses to an ear of the subject, wherein the one or more electrical pulses are sufficient to activate the one or more neuromodulatory systems (e.g., the noradrenergic system) of the subject, wherein activation (optionally, acute activation) of the one or more neuromodulatory systems extinguishes conditioned fears through repeated reminders of traumatic events. In some aspects, exposure therapy can extinguish conditioned fears through repeated reminders of traumatic events. Extinction of the conditioned fear can depend on the consolidation of new memories made with these exposures.

Disclosed are methods of treating a disease or disorder in a subject comprising applying one or more electrical pulses to an ear of the subject, wherein the one or more electrical pulses are sufficient to activate the one or more neuromodulatory systems (e.g., the noradrenergic system) of the subject, wherein activation (optionally, acute activation) of the one or more neuromodulatory systems improves consolidation and maintenance of the extinction memory.

Disclosed are methods of ameliorating a symptom associated with a psychiatric disorder, neurological disorder, or chronic inflammatory disorder in a subject comprising applying one or more electrical pulses to an ear of the subject, wherein the one or more electrical pulses are sufficient to ameliorate a symptom associated with a psychiatric disorder, neurological disorder, chronic inflammatory, or other disorder in a subject. In some aspects, the psychiatric disorder, neurological disorder, chronic inflammatory, or other disorder are any of those disclosed herein. In some aspects, a symptom associated with a psychiatric disorder, neurological disorder, chronic inflammatory, or other disorder can be, but is not limited to, seizures, generalized perceptions of pain, feelings of sadness and/or hopelessness, irritability, loss of interest, decrease sleep disturbance, increase appetite, enhanced cognition, decrease feelings of worthlessness, thoughts of self-harm/suicide, intrusive thoughts/flashbacks, irritability and hypervigilance, altered motor function. Thus, any one or more of the disclosed symptoms can be ameliorated

Disclosed are methods to support learning in a subject in need thereof comprising applying one or more electrical pulses to an ear of the subject, wherein the one or more electrical pulses are sufficient to activate (optionally, acutely activate) the one or more neuromodulatory systems (e.g., the noradrenergic system) of the subject in need thereof, wherein the subject in need thereof has enhanced learning. In some aspects, a subject in need thereof can be a healthy individual. In some aspects, a subject in need thereof can be a subject that has suffered a stroke. In some aspects, a subject in need thereof can be a subject having a psychiatric disorder, neurologic disorder, chronic inflammatory disorder, or other disorder. In some aspects, the psychiatric disorder, neurological disorder, chronic inflammatory, or other disorder are any of those disclosed herein.

In some aspects, “electrical pulse” can be used interchangeably with “electrical impulse.” In some aspects, one or more electrical pulses is the same as one or more pulses of electrical current. In some aspects, one or more electrical pulses comprise a stimulation train and several stimulation trains can be applied in the context of a single treatment or application. In some aspects, a stimulation train is a group of electrical impulses that are close together in time.

In some aspects in any of the disclosed methods, the one or more electrical pulses are applied unilaterally to the subject. In some aspects, the one or more electrical pulses are applied only to the external ear of the subject. In some aspects, one or more electrical pulses can be applied to the cymba and cavum concha of a subject. In some aspects, one or more pulses can be applied to the tragus of a subject. In some aspects, the one or more electrical pulses are applied only to the left ear of the subject. In some aspects, the one or more electrical pulses are applied only to the external ear of the left ear of the subject. In some aspects, the one or more electrical pulses are applied only to the ear canal of the left ear of the subject. Thus, in some aspects, one or more electrical pulses are not applied to the right ear of the subject. In some aspects, the one or more electrical pulses are applied only to the right ear of the subject. In some aspects, the one or more electrical pulses are applied only to the external ear of the subject. In some aspects, the one or more electrical pulses are applied only to the ear canal of the right ear of the subject. Thus, in some aspects, one or more electrical pulses are not applied to the left ear of the subject.

In some aspects in any of the disclosed methods, the one or more electrical pulses are applied bilaterally to the subject. Thus, in some aspects, the one or more electrical pulses are applied to both the right and left ear of the subject.

In some aspects, applying one or more electrical pulses to the vagus nerve of the subject comprises applying according to a treatment paradigm. A treatment paradigm is a schedule or program of treatments designed for treating a disease or disorder or treating a symptom of a disease or disorder. In some aspects, the treatment paradigm comprises a one or more electrical pulses for a time period of about 1 ms to 1000 ms. In some aspects, the treatment paradigm comprises one or more electrical pulses for a time period of about 500 ms to 800 ms. In some aspects, the treatment paradigm comprises one or more electrical pulses for a time period of about 100, 150, 200, 250, 300, 350, 400, 450, 500, 550, 600, 650, 700, 750, or 800 ms. In some aspects, the treatment paradigm comprises continuously applying the electrical pulses for a time period of about 1 ms to 1000 ms. In some aspects, the treatment paradigm comprises one or more electrical pulses for a time period of about 30 seconds to 30 minutes. In some aspects, the treatment paradigm comprises one or more electrical pulses for a time period of about 1, 2, 3, 4, 5, 6, 7, 8, 9, 10, 15, 20, 25, or 30 minutes. In some aspects, a treatment paradigm comprises one or more electrical pulses comprising a stimulation train or several stimulation trains in a single treatment. In some aspects, the one or more electrical pulses coincides with an environmental event or a behavior therefore helping in learning the environmental event or behavior.

In some aspects, applying one or more electrical pulses occurs with a pulse width of 100 μs and 1,000 μs. In some aspects, the one or more electrical pulses have a pulse width of between 100 μs and 300 μs, between 200 μs and 400 μs, between 250 μs and 350 μs, or between 300 μs and 400 μs. In some aspects, the one or more electrical pulses have a pulse width of 100, 150, 200, 250, 300, 350, 400, 450, or 500 μs. In some aspects, the one or more electrical pulses have a pulse width of 300 μs.

In some aspects, the one or more electrical pulses have a frequency of between 1 Hz and 10,000 Hz. In some aspects, the one or more electrical pulses have a frequency of between 100 Hz and 300 Hz, between 200 Hz and 400 Hz, between 250 Hz and 350 Hz, between 300 Hz and 400 Hz. In some aspects, the one or more electrical pulses have a frequency of 100, 150, 200, 250, 300, 350, 400, 450, or 500 Hz. In some aspects, the one or more electrical pulses have a frequency of 300 Hz.

In some aspects, the current amplitude can be normalized on a subject by subject basis. Because the current amplitude is determined based on perceptual threshold and each subject can have a slightly varied perceptual threshold, the current amplitude is not necessarily a set value. Although the current amplitude can vary subject to subject, in some aspects there can be a critical window since too much neurotransmitter, which is released upon auricular stimulation, can interfere with performance. Thus, the current amplitude can be titrated relative to perceptual threshold in a way that promotes learning.

In some aspects, the treatment paradigm is applied once daily. In some aspects, the treatment paradigm is applied at least twice daily. In some aspects, each treatment paradigm is applied within 5 minutes of each other. In some aspects, the treatment paradigm is applied once a week. In some aspects, the treatment paradigm is applied once a month.

In some aspects, activating (optionally, acutely activating) the one or more neuromodulatory systems (e.g., the noradrenergic system) comprises stimulating the vagus nerve of the subject. In some aspects, stimulating the vagus nerve comprises taVNS.

In some aspects of the disclosed methods, the one or more electrical pulses to an ear of the subject are applied simultaneously with a behavior, environmental, or task-related event to be learned or unlearned. Thus, in some aspects, the one or more electrical pulses to an ear of the subject enhance the learning or unlearning of the behavior, environmental, or task-related event administered or provided simultaneously.

In some aspects, the disclosed methods to support learning can be used to support learning of any type of skill or activity. In some aspects, the disclosed methods to support learning can be used to support learning how to move one or more body parts; learning how to associate two or more items; learning daily activities such as cooking, cleaning, and self-grooming; learning fundamental motor skills such as walking, running, jumping; learning sports skills such skiing, kayaking, boarding, golfing; learning language skills such as producing speech, understanding speech, reading, writing, singing; and learning other daily skills such as driving a vehicle, meditating, mathematical computation. In some aspects, the disclosed methods to support learning can be used to support learning any behavior whether it is a learned behavior or innate behavior. For example, an innate behavior can be refined as a process of neural maturation and therefore can be learned in the disclosed methods. In some aspects, environmental events refer to the subjective association one has to perceptual phenomena and can vary based on personal experience.

### D. Exemplary Stimulation Apparatus

Currently, there are multiple taVNS devices available on the market and likely many more in development. The electrode interface associated with most devices tends to target the conchae or tragus of the external ear and in a “one-size-fits-all” manner. Although the conchae and tragus are two putative landmarks of auricular innervation, various aspects of the external auditory meatus (i.e., ear canal) also have been implicated by human cadaver and functional imaging studies. Multiple factors complicate the determination of which landmark to target. Specifically, cutaneous distributions of a given nerve root can vary between individuals, cross-communication between fibers belonging to different nerves is plausible, and dermatomes are unlikely to exhibit rigid boundaries. Moreover, the extent and complexity of vagal innervation in each putative landmark on the external ear is subject to inter-individual variation. For instance, aside from the auricular branch of the vagus nerve, the concha is partly innervated by the auriculotemporal, facial, greater auricular, and lesser occipital nerves. Available anatomical evidence from cadavers, although limited, indicates that the posterior wall of the ear canal is innervated by the auricular vagus nerve, but the auriculotemporal and greater auricular nerves also are known to innervate canal walls. Consequently, uncertainty exists in the field regarding where and how to stimulate. In particular, the question of where to stimulate remains largely has been unresolved. The uniqueness of external ear structure in humans is, in fact, comparable to that of the fingerprint. Given the lack of knowledge in this regard and situations in which targeting a single landmark with taVNS is not a viable option to confer therapeutic benefit, disclosed herein is an electrode interface that is capable of targeting a plurality of putative sites of auricular vagus nerve innervation while also accounting for the unique external ear structure of the end user.

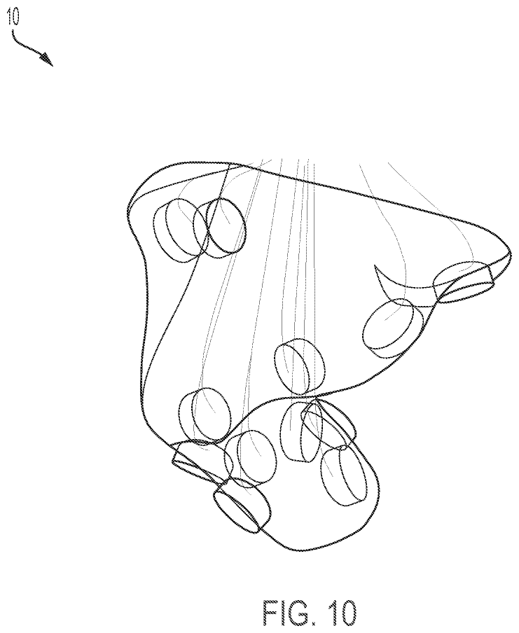

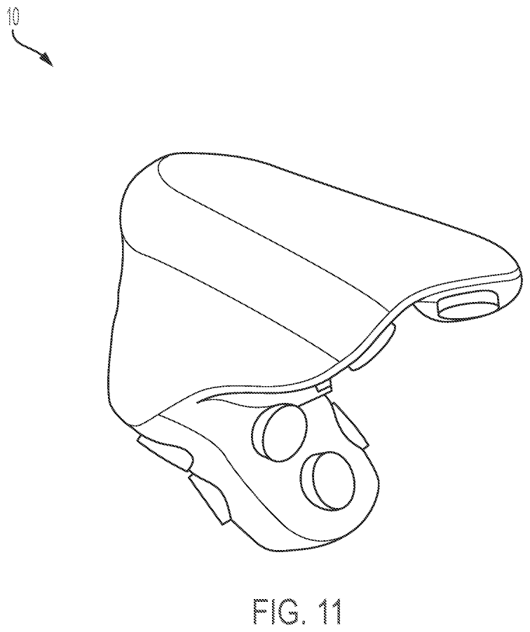

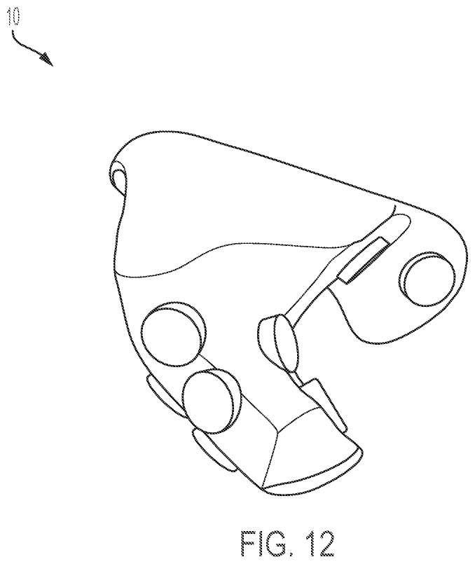

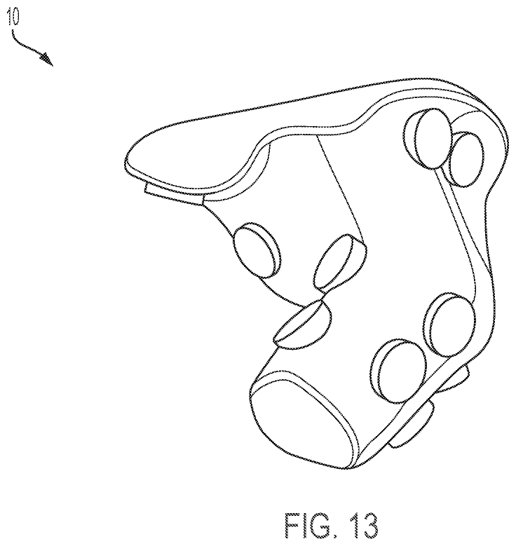

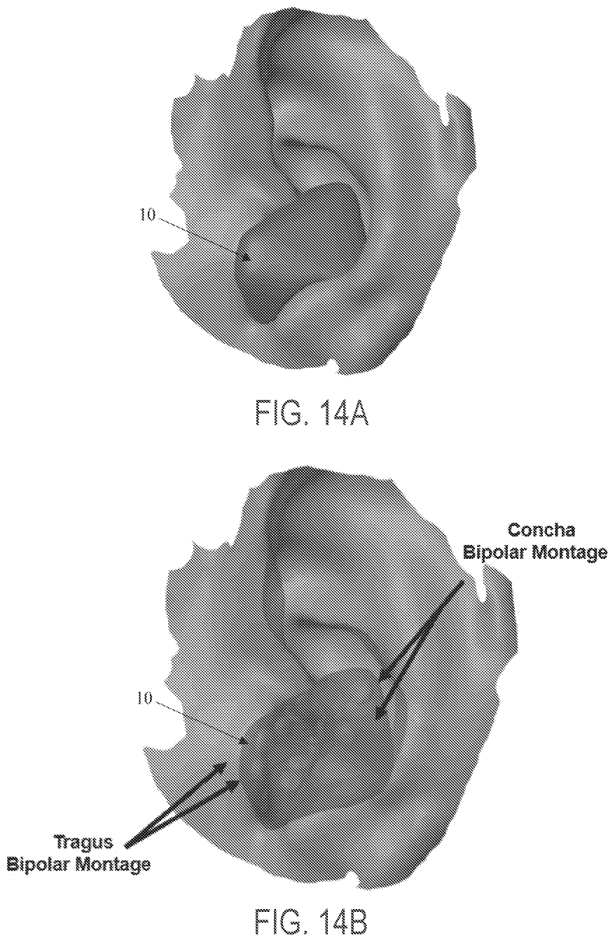

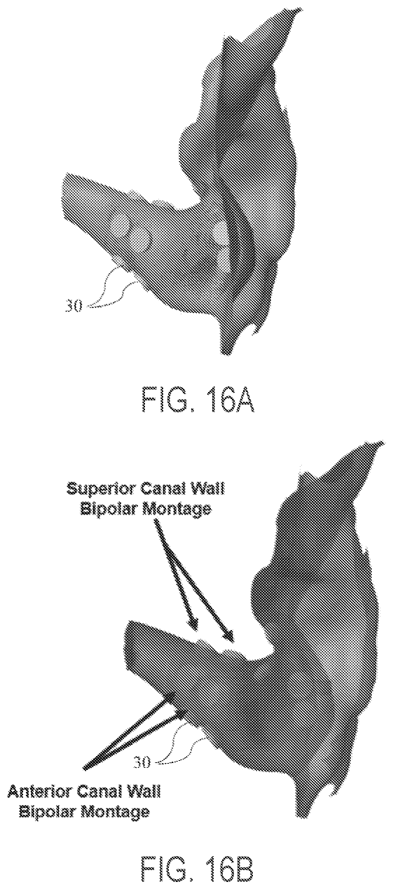

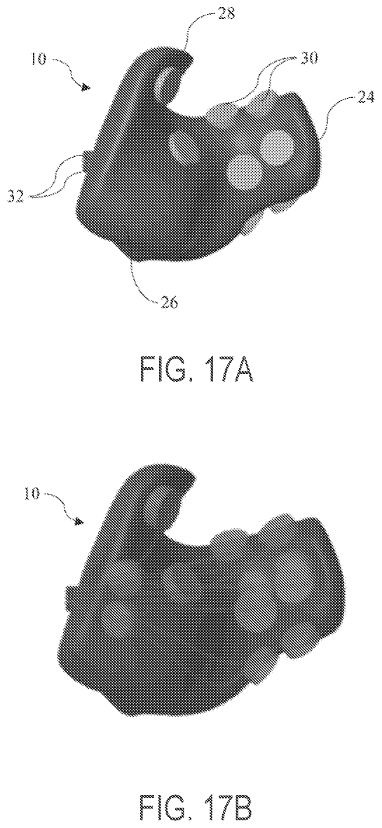

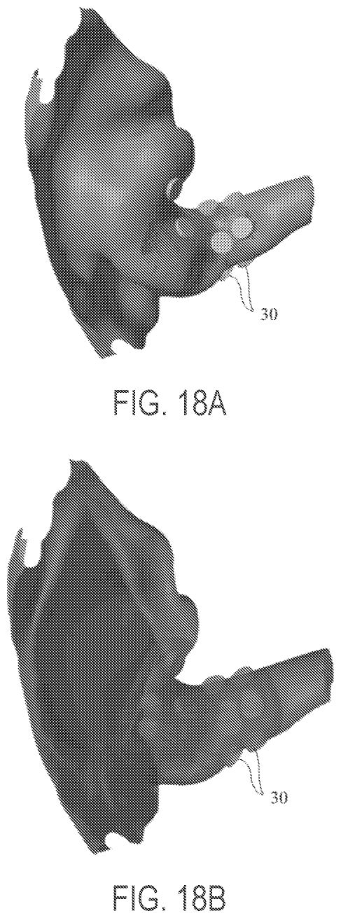

Referring to FIGS. 10-18B, an apparatus 10 can be configured to provide stimulation as disclosed herein. As should be understood, the treatments described herein should not be limited to use with the particular embodiments of the apparatus 10 described and depicted herein. Rather, the apparatus 10 merely provides an exemplary embodiment for provision of such treatments.

The apparatus 10 can comprise a body 20 that is configured to be at least partially receivable into an ear canal. For example, the body 20 can be shaped for complementary receipt into an ear canal. In these aspects, the body 20 can have an outer surface 22 that is shaped based on a mold or a scan of an ear canal. The body 20 can cover a contiguous area of the external ear innervated by the auricular vagus nerve.

The body 20 can comprise a first portion 24 that is configured to be received into the ear. The outer surface 22 of the body 20 along the first portion 24 can be user-specific and be complementary to the surfaces of the ear canal. A second portion 26 of the body 20 can extend outwardly from the ear canal. The second portion 26 can comprise a tragus protrusion 28 that is configured to contact the tragus of the ear. The tragus protrusion 28 can have at least one electrode coupled thereto.

As should be understood, conventional hearing aids are configured to be low profile for aesthetic purposes and otherwise to remain fixed. The disclosed body 20, in contrast, can extend outwardly from the ear canal. For example, in some aspects, the second portion 26 of the body 20 is configured to extend outwardly from the ear canal by at least 4 mm, at least 5 mm, at least 6 mm, at least 7 mm, at least 8 mm, at least 9 mm, or at least 1 cm when the first portion 24 of the body 20 is fully received within the ear canal.

A plurality of electrodes 30 can be coupled to the body 20. In some aspects, the plurality of electrodes 30 can be coupled to an outer surface 22 of the body 20. In some aspects, the plurality of electrodes 30 can be at least partly recessed within the body. In some aspects, the plurality of electrodes 30 can extend outwardly from the outer surface of the body.

In some aspects, the apparatus 10 can comprise pairs of electrodes 30 that are positioned at different areas of the ear. The electrodes 30 within a given pair can have a spacing from each other ranging from about 0.5 to about 1.0 mm. The pairs can be centered at different anatomical landmarks (e.g., conchae, tragus, and/or one or more areas of walls of the ear canal). Thus, it is contemplated that a particular landmark can be positioned between the first and second electrodes of a respective electrode pair. Optionally, the electrodes can comprise sintered disc electrodes. The electrodes 30 can be embedded in the mold. Optionally, the electrodes 30 can have a diameter from about 2 mm to about 6 mm, or about 4 mm.

In some aspects, the first portion 24 of the body 20 can be elongated (have an elongated profile) along a first axis 25. The first axis 25 can extend centrally through the first portion 24 of the body 20. In some optional aspects, the plurality of electrodes 30 comprise pairs of electrodes 30 that are spaced from each other along the first axis 25. In further aspects, the pairs of electrodes 30 can be circumferentially spaced about the first axis 25.

In some aspects, the apparatus 10 can have between 6 electrodes and 12 electrodes coupled to the first portion 24 of the body 20. For example, in some aspects, the apparatus 10 can have 8 electrodes coupled to the first portion 24 of the body 20. In these aspects, the apparatus 10 can comprise four pairs of electrodes 30 that are configured to stimulate four different areas of the ear canal. In some optional aspects, the pairs of electrodes can be equally circumferentially spaced about the first portion 24 of the body 20. In other aspects, the pairs of electrodes can be unequally circumferentially spaced (e.g., with differences in circumferential spacing that are greater than 5% to 10% of the circumference) about the first portion 24 of the body 20.

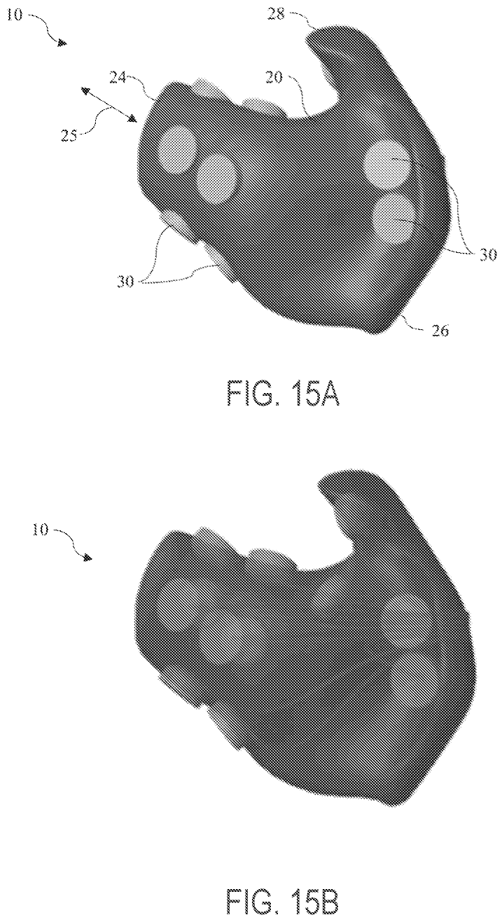

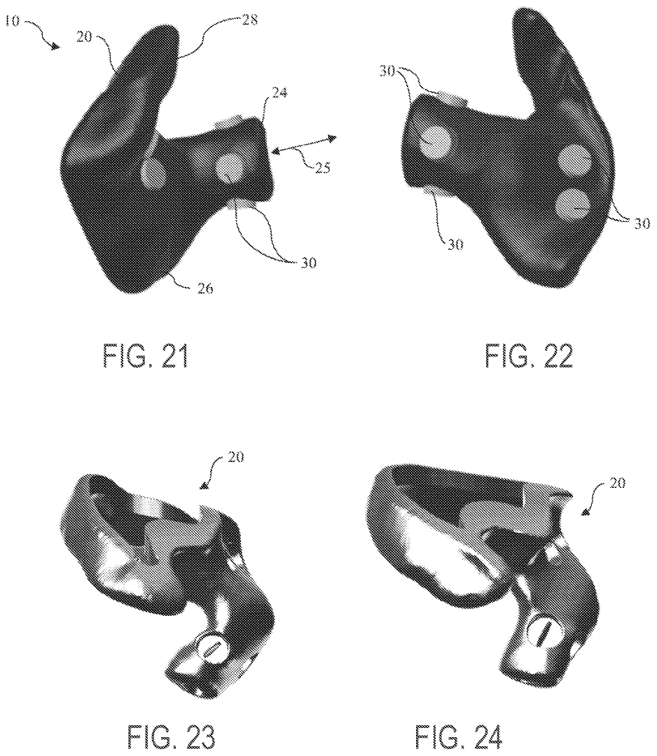

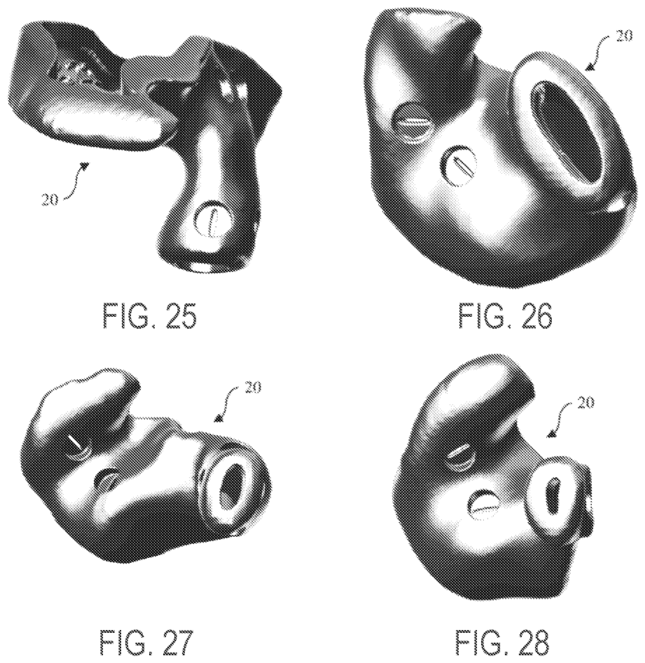

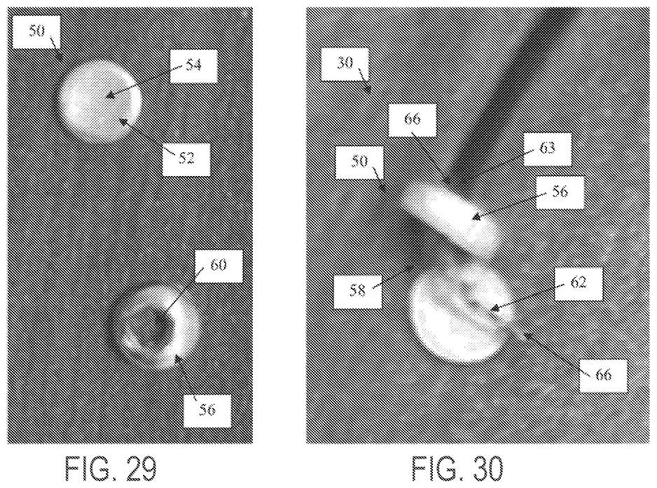

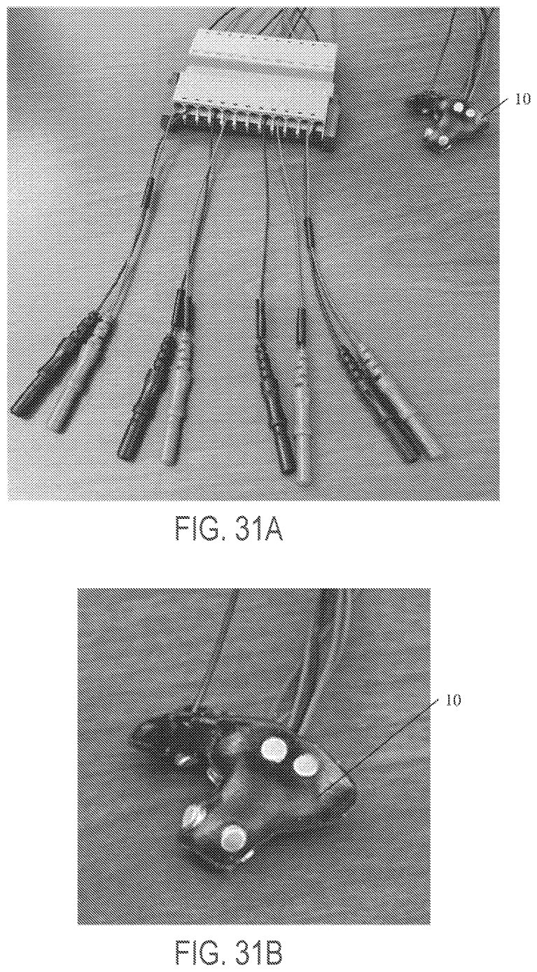

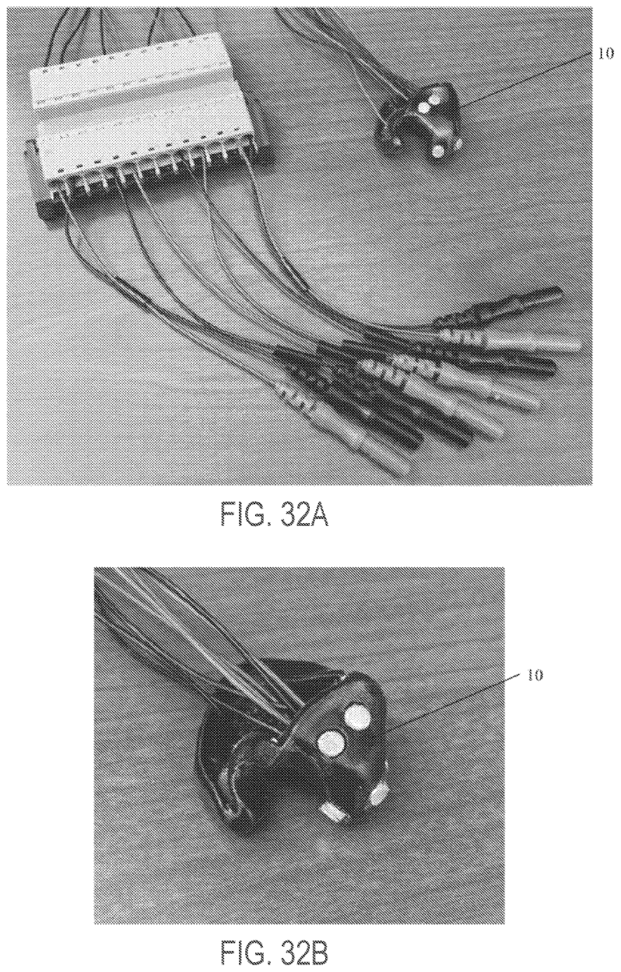

Referring also to FIGS. 21-22, 31A, 31B, 32A, and 32B, in some aspects, rather than providing pairs of electrodes at different regions of the body 20, the apparatus 10 can comprise individual electrodes at different regions of the first portion 24 of the body 20 (such that only one electrode is provided at each respective region of the first portion 24 of the body 20). This can contrast to, for example, pairs of electrodes spaced along the first axis 25 in each region, as illustrated in FIGS. 10-18B. Thus, in some aspects, respective axes parallel to the first axis 25 that extend through each electrode 30 on the first portion 24 of the body 20 can extend through only a single respective electrode. For example, a front region of the first portion 24 and a rear region of the first portion 24 can each have only a single electrode 30. In further aspects, a top region of the first portion 24 and a bottom region of the first portion 24 can each have only a single electrode 30. Accordingly, in some aspects, the apparatus can have four electrodes coupled to the body. It is contemplated that stimulation can be provided between two electrodes (e.g., two adjacent electrodes) positioned on different regions of the first portion 24 of the body. Optionally, it is contemplated that one or more (optionally, all) of the different regions where respective individual electrodes are positioned on the first portion 24 of the body 20 can correspond to or be in proximity to locations of anatomical landmarks as further disclosed herein. It is contemplated that an apparatus having single electrodes at different regions of the first portion 24 of the body 20, in contrast to pairs of electrodes (illustrated in FIG. 15A), can be easier to manufacture and more easily received within the ear of the patient. In some optional aspects, the first portion 24 of the body 20 can have a single electrode coupled thereto. In various aspects, the first portion 24 of the body 20 can have exactly two, exactly three, exactly four, or more electrodes coupled thereto. In some aspects, the electrodes 30 can be equally circumferential spaced or substantially equally circumferentially spaced (e.g., with differences in circumferential spacing that are within 5% to 10% of the circumference) about the first portion 24 of the body 20. In other aspects, the electrodes 30 can be unequally circumferentially spaced (e.g., with differences in circumferential spacing that are greater than 5% to 10% of the circumference) about the first portion 24 of the body 20.

In some aspects, the second portion 26 of the body 20 can be configured to contact surfaces of the concha of the ear. At least one electrode 30 can be coupled to the second portion 26 of the body 20 and can be configured to contact the concha of the ear. In some aspects, the apparatus 10 can have at least one, at least two, or exactly two electrodes that are coupled to the second portion of the body in position to contact the concha of the ear. For example, the pair can be used to provide stimulation to the concha.

In some aspects, the apparatus 10 can have a single electrode coupled to the tragus protrusion 28. In some aspects, the apparatus 10 can have at least one electrode 30 (optionally, a single electrode) that is positioned on the second portion 26 of the body and is configured to contact a side of the tragus opposite the electrode on the tragus protrusion 28. Accordingly, in some aspects, a pair of electrodes (with one electrode on each opposing side of the tragus) can be used to provide stimulation to the tragus from both opposed sides.

In some aspects, the plurality of electrodes 30 can comprise adjacent electrodes that are spaced apart by a spacing from 0.2 mm to about 5 mm (e.g., from 0.5 mm to about 1.0 mm).

In some aspects, the body 30 can be flexible. For example, in some aspects, the body can comprise biocompatible elastomer (e.g., silicone).

The body 20 can be configured to permit sound to travel therethrough. For example, the body can define a conduit therethrough. In other aspects, the body 20 can have a porosity (e.g., an open porosity) that permits sound conduction therethrough. For example, in some aspects, the body can comprise open cell foam. In some aspects, the body 20 can be hollow so as to not occlude hearing and to enable passage of wires/electrode leads.

In some aspects, the apparatus 10 can comprise a plurality of electrical leads 32 that couple to and extend from respective electrodes 30 of the plurality of electrodes. Optionally, the electrical leads 32 can extend through the conduit that extends through the body.

In some aspects, a method of making the apparatus 10 can comprise scanning an ear of a user (for example, by three-dimensional scanning as is known in the art). In other aspects, the method can comprise taking a mold of the ear of the user. The method can further comprise determining at least a portion of a shape of the outer surface 22 of the body 20 based on the scan or the mold of the ear. For example, a portion of the outer surface 22 of the body can match (e.g., be complementary to) a surface of the scan or the mold.

In some exemplary aspects, the mold can be developed based on a 3-dimensional scan of the external ear obtained by a commercially available system (Otoscan®), Natus Medical Incorporated). Coordinates from the scan can be uploaded to computer aided design (CAD) software to generate a rendering of the ear for positioning electrodes and/or fabricating the mold.

In some optional aspects, the body 20 can be 3D printed. In other aspects, the body 20 can be cast in a mold. In some optional aspects, the mold for the body 20 can be 3D printed. In some optional aspects, the mold can be formed from a 3D printed element having the shape of the body.

In some aspects, the body 20 can comprise, or be made of, an elastomer material that is biocompatible. The body 20 can have a durometer from about 30 to about 95 (e.g., from about 60 to about 80, or about 70).

In exemplary aspects, the body 20 can cover an entire surface area that includes all putative sites of auricular innervation (e.g., the ear canal, tragus, and conchae), extending from inside the canal out through the concha bowl of the ear of the subject.

The body 20 can be scaled down or up from the scan to improve fit. For example, in some aspects, the body 20 can be scaled down 0.1 mm to about 0.5 mm from the scan. In other aspects, the body 20 can generally fit the true size obtained during the scan. Sizing down can be used when the canal is narrow, causing electrodes to protrude more from cavities in the canal.

The body 20 can define cavities in different regions for receiving electrodes 30. Optionally, the cavities can have a shape of a truncated cone. In some aspects, cavity depth and wall thickness can depend on cavity location. For example, the cavities in the body for positioning at the conchae can have a depth of about 1.5 mm; the cavities in the body for positioning at the tragus can have a depth ranging from about 1 mm to about 1.25 mm; and the cavities in the body for positioning at the canal can have a depth from about 0.75 mm to 1.25 mm. The body can have a wall thickness from about 0.4 mm to about 0.6 mm.

The body can receive the electrodes via press-fit into the cavities. Accordingly, the cavities can have dimensions that are less than those of the electrodes. For example, when using electrodes having outer diameters of 3.7 mm, the base of each cavity can range from approximately 3.4-3.7 mm, and the opening of each cavity can be about 3.55±0.05 mm. The difference in cavity base and opening provides for added stability of electrodes in the cavity and is intended to reduce displacement. This can allow for secure retention of the electrode within the cavity. For example, in some aspects, the base of the cavity can be wider than the opening of the cavity.

Advantageously, the disclosed apparatus 10 can position electrodes 30 simultaneously at multiple stimulation locations within the ear of the user. Accordingly, the apparatus 10 can, without adjustment or modification to the apparatus, permit stimulation at various locations as well as adjustment to stimulation routines to improve desired outcomes. In this way, the apparatus 10 can be adaptable for use with a subject, regardless of the positions of the nerves of the subject relative to the geometry of the ear.

In some aspects, brief (e.g., less than one second) pulse trains can be applied through a bipolar montage on the external ear to activate (optionally, acutely activate) neuromodulatory systems implicated in the therapeutic effects of VNS. Various effects and interactions of stimulation location, pulse amplitude, and pulse frequency on one or more biomarker of the targeted neuromodulatory systems can be shown. The disclosed apparatus can enhance the ability to more effectively examine physiological effects of stimulating the various putative landmarks of auricular vagus nerve innervation. In addition to providing therapy, the disclosed apparatus can be used to study therapeutic effects in humans by tracking one or more biomarkers.

In some aspects, a stimulation routine can comprise using at least a first electrode to apply stimulation. The stimulation routine can be adjusted to provide stimulation via a second electrode based on at least one measured outcome.

It should be understood that the disclosed apparatuses and methods used herein can be used for any therapeutic applications and are not limited to the therapeutic applications disclosed herein.

Referring to FIGS. 29 and 30, it is further contemplated that the electrode 30 can comprise a housing 50, the housing having a first housing portion 52 defining a skin contact surface 54 of the electrode and a second housing portion 56 that cooperates with the first housing portion to define an enclosure 58. The second housing portion can define an opening 60 (e.g., optionally, a central opening). A conductor 62 (e.g., a wire) can extend through the opening 60. The conductor 62 can have a first end 64 that is soldered to the first housing portion 52 and the second housing portion 56 so that solder 66 is contained within the enclosure 58 of the housing 50. In some aspects, to make the electrode 30, the second portion 56 of the housing 50 can first be soldered to the conductor 62, and the first portion 52 can subsequently be soldered to the conductor 62.

It is contemplated that the housing 50 can define specific outer geometry that permits the electrode 30 to be receivable within the body 20. For example, the skin contact surface 54 of the electrodes 30 can have a major dimension (e.g., diameter) of about 4 mm, or less than 4 mm (e.g., about 3.7 mm, or less than 3.7 mm, or between 1 mm and 4 mm, or between 3 mm and 4 mm). The electrodes can have a depth from about 1 mm to about 3 mm (e.g., about 2 mm). Conventional off-the-shelf electrodes, particularly off-the-shelf electrodes having such a small major dimension as, for example, 4 mm, have inconsistent exterior dimensions. When setting the electrodes 30 within the body 20, if the electrodes are not an exact fit, the electrodes will be uncomfortable, can fall out of the body, or are otherwise not be suitable for use. Accordingly, the disclosed electrodes can have defined geometry that can provide improved compatibility with the apparatus 10.

It is further contemplated that, by extending the conductor 62 through the opening 60 of the second portion 56 of the housing 50, the conductor can extend away from the skin contact surface 54 of the electrode 30. This can contrast to electrodes in which the conductor extends from the side of the electrode and is less suited for receipt within the body 20.

Optionally, the first housing portion 52 can have a rounded peripheral edge 53 that surrounds the contact surface 54. In some aspects, the housing 50 can comprise silver (e.g., sterling silver). In various aspects, the electrodes can be wet electrodes or dry electrodes.

In some optional aspects, a portion 63 of the conductor 62 that extends through the central opening can be encapsulated in polymer 66. Optionally, the polymer 66 can be silicone. The polymer 66 can provide strain relief as well as electrical insulation while still allowing flexibility of the conductor 62.

In exemplary optional aspects, the electrodes can be made of sterling silver and be from 3 to 4 mm (about 3.7 mm) in diameter and from 1.5 to 2.5 mm (about 2 mm) in depth.

In exemplary aspects, during manufacture of an electrode, two separate discs can be cut, and a wire can be clamped and soldered to both discs through a hole drilled into the disc facing the base of the cavity.

In use, it is contemplated that the disclosed apparatus can provide improved electrode fit (within cavities), improved comfort on the subject's skin, improved contact with the subject's skin, and/or increased flexibility of the wires/electrode leads relative to their points of contact with the electrode discs. It is contemplated that the increased flexibility of wires can be particularly beneficial because space can be limited inside the mold, particularly for subjects with narrow ear canals.

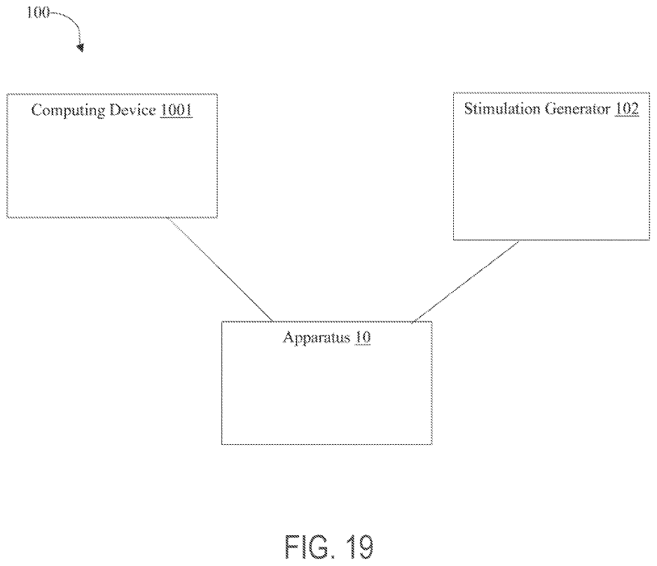

Referring also to FIG. 19, a system 100 can comprise an apparatus 10 and a stimulation generator 102. The stimulation generator 102 can be in communication with the first electrical conductor 24 and the second electrical conductor 26 of the apparatus 10. The stimulation generator 102 can be configured to generate current for providing electrical stimulation through or between the first and second electrodes 20, 22 of the apparatus 10.

Optionally, the system 100 can comprise a computing device (e.g., computing device 1001 as further disclosed herein). The computing device can comprise at one or more processors (e.g., processor 1003) and a memory (e.g., mass storage device 1004) in communication with the processor(s). The memory can comprise instructions that, when executed by the processor(s), causes the processor(s) to receive data from the apparatus 10. The computing device can further comprise a display device (e.g., display device 1011) and an input device (e.g., input device 1020). The memory can comprise instructions that, when executed by the processor(s), causes the processor(s) to cause the display device to display the data received from the apparatus. In yet further aspects, the computing device can receive an input from a clinician to enable the clinician to adjust one or more parameters of the stimulation generator (e.g., pulse frequency, pulse amplitude, pulse width, or duration of a stimulation session) based on the received data.

In further aspects, memory can comprise instructions that, when executed by the processor(s), causes the processor(s) to automatically adjust the one or more parameters of the stimulation generator based on the received data from the apparatus 10.

### E. Computing Device

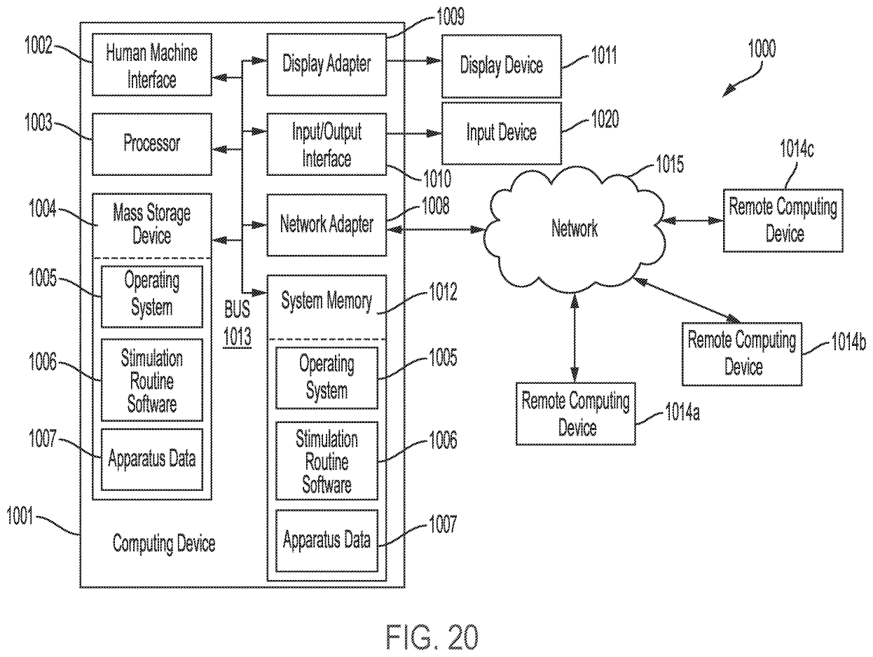

FIG. 20 shows a computing system 1000 including an exemplary configuration of a computing device 1001 for use with the stimulation system 100. In some aspects, the computing device 1001 can be part of a network. In further aspects, it is contemplated that a separate computing device, such as, for example, a tablet, laptop, or desktop computer can communicate with the system 10 and can enable the operator to interface with the system 10.

The computing device 1001 may comprise one or more processors 1003, a system memory 1012, and a bus 1013 that couples various components of the computing device 1001 including the one or more processors 1003 to the system memory 1012. In the case of multiple processors 1003, the computing device 1001 may utilize parallel computing.

The bus 1013 may comprise one or more of several possible types of bus structures, such as a memory bus, memory controller, a peripheral bus, an accelerated graphics port, and a processor or local bus using any of a variety of bus architectures.

The computing device 1001 may operate on and/or comprise a variety of computer readable media (e.g., non-transitory). Computer readable media may be any available media that is accessible by the computing device 1001 and comprises, non-transitory, volatile and/or non-volatile media, removable and non-removable media. The system memory 1012 has computer readable media in the form of volatile memory, such as random access memory (RAM), and/or non-volatile memory, such as read only memory (ROM). The system memory 1012 may store data such as apparatus data 1007 (i.e., data from signals received by the electrodes) and/or program modules such as operating system 1005 and stimulation routine software 1006 that are accessible to and/or are operated on by the one or more processors 1003.

The computing device 1001 may also comprise other removable/non-removable, volatile/non-volatile computer storage media. The mass storage device 1004 may provide non-volatile storage of computer code, computer readable instructions, data structures, program modules, and other data for the computing device 1001. The mass storage device 1004 may be a hard disk, a removable magnetic disk, a removable optical disk, magnetic cassettes or other magnetic storage devices, flash memory cards, CD-ROM, digital versatile disks (DVD) or other optical storage, random access memories (RAM), read only memories (ROM), electrically erasable programmable read-only memory (EEPROM), and the like.

Any number of program modules may be stored on the mass storage device 1004. An operating system 1005 and stimulation routine software 1006 may be stored on the mass storage device 1004. One or more of the operating system 1005 and stimulation routine software 1006 (or some combination thereof) may comprise program modules and the stimulation routine software 1006. The apparatus data 1007 may also be stored on the mass storage device 1004. The apparatus data 1007 may be stored in any of one or more databases known in the art. The databases may be centralized or distributed across multiple locations within the network 1015.

A user may enter commands and information into the computing device 1001 using an input device 1020. Such input devices comprise, but are not limited to, a keyboard, pointing device (e.g., a computer mouse, remote control), a microphone, a joystick, a scanner, tactile input devices such as gloves, and other body coverings, motion sensor, and the like. These and other input devices may be connected to the one or more processors 1003 using a human machine interface 1002 that is coupled to the bus 1013, but may be connected by other interface and bus structures, such as a parallel port, game port, an IEEE 1394 Port (also known as a Firewire port), a serial port, network adapter 1008, and/or a universal serial bus (USB).

A display device 1011 may also be connected to the bus 1013 using an interface, such as a display adapter 1009. It is contemplated that the computing device 1001 may have more than one display adapter 1009 and the computing device 1001 may have more than one display device 1011. A display device 1011 may be a monitor, an LCD (Liquid Crystal Display), light emitting diode (LED) display, television, smart lens, smart glass, and/or a projector. In addition to the display device 1011, other output peripheral devices may comprise components such as speakers (not shown) and a printer (not shown) which may be connected to the computing device 1001 using Input/Output Interface 1010. Any step and/or result of the methods may be output (or caused to be output) in any form to an output device. Such output may be any form of visual representation, including, but not limited to, textual, graphical, animation, audio, tactile, and the like. The display device 1011 and computing device 1001 may be part of one device, or separate devices.

The computing device 1001 may operate in a networked environment using logical connections to one or more remote computing devices 1014a,b,c. A remote computing device 1014a,b,c may be a personal computer, computing station (e.g., workstation), portable computer (e.g., laptop, mobile phone, tablet device), smart device (e.g., smartphone, smart watch, activity tracker, smart apparel, smart accessory), security and/or monitoring device, a server, a router, a network computer, a peer device, edge device or other common network node, and so on. Logical connections between the computing device 1001 and a remote computing device 1014a,b,c may be made using a network 1015, such as a local area network (LAN) and/or a general wide area network (WAN), or a Cloud-based network. Such network connections may be through a network adapter 1008. A network adapter 1008 may be implemented in both wired and wireless environments. Such networking environments are conventional and commonplace in dwellings, offices, enterprise-wide computer networks, intranets, and the Internet. It is contemplated that the remote computing devices 1014a,b,c can optionally have some or all of the components disclosed as being part of computing device 1001. In various further aspects, it is contemplated that some or all aspects of data processing described herein can be performed via cloud computing on one or more servers or other remote computing devices. Accordingly, at least a portion of the system 1000 can be configured with internet connectivity.

### E. Track and Map Responses

In some aspects, the system described herein can be used when tracking and mapping user responses. For example, a system can be configured to provide VNS only during specific durations, such as, for example, when a user is performing a desired task.

Such tracking and mapping of user responses can be beneficial for analyzing efficacy in users such as stroke survivors. For example, a computing device can receive feedback such as, for example, a force applied to a force sensor. A user can be instructed (e.g., via a display device) to apply a select force, within maximum and minimum thresholds. The computing device can, based on the feedback from the force sensor, whether the user applied the select force within the thresholds for a predetermined duration and, in response, provide VNS (e.g., via the system 100). Similar VNS delivery based on a tracked metric can be used for PTSD treatment or other diseases or disorders.

### EXAMPLES

### 1. Background

Vagus nerve stimulation (VNS) is FDA approved for treatment of depression and epilepsy. Human trials are examining its therapeutic potential in multiple chronic inflammatory disorders including, but not limited to fibromyalgia, migraine headaches, and obesity. Preclinical animal work is also showing promise for treating psychiatric disorders such as post-traumatic stress disorder and motor impairments resulting from neurological injury to the corticospinal tract. In post-traumatic stress disorder, exposure therapy works to extinguish conditioned fears through repeated reminders of traumatic events. Extinction of the conditioned fear depends on the consolidation of new memories made with these exposures. VNS is being explored as an adjunct therapy to improve consolidation and maintenance of the extinction memory. The idea is that under stressful conditions, the vagus nerve signals the brain to facilitate the storage of new memories while, as part of the parasympathetic nervous system, it slows the sympathetic response.

Traumatic events lead to activation of the sympathetic nervous system via the fight-or-flight response. The resulting peripheral changes, such as increased heart and respiration rate, occur during the memory consolidation window and are associated with enhanced memory storage. However, epinephrine does not readily cross the blood-brain barrier. Rather, it binds to beta-adrenergic receptors on the vagus nerve, which then activate brainstem nuclei to release norepinephrine throughout the brain, leading to storage of a newly acquired memory. VNS promotes brain plasticity but bypasses the peripheral fight-or-flight response. Rather, VNS engages the parasympathetic nervous system, slowing heart rate and increasing gut motility. Therefore, pairing VNS with exposure therapy has the potential to strengthen the extinction memory by tapping into mechanisms that enhance storage of the traumatic memory without the requirement of a sympathetic stress response

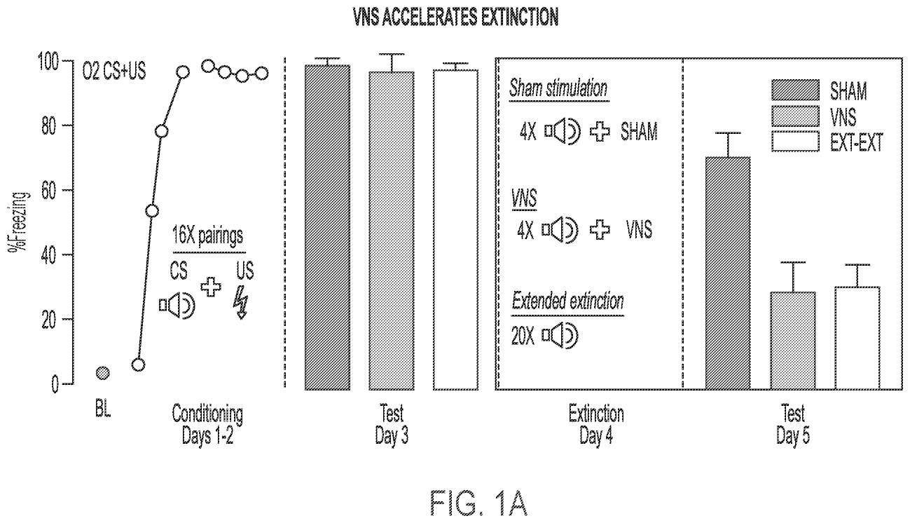

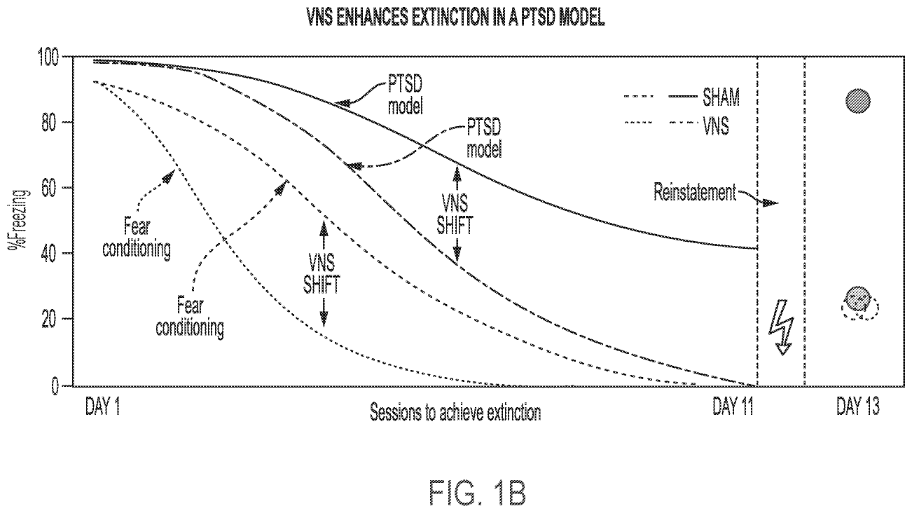

Preclinical animal work is providing support for this theory (FIG. 1). In fear conditioned rats undergoing extinction training, VNS is temporally paired with exposures to a conditioned stimulus (ie, an auditory tone previously paired with electrical shocks to the limb).

As shown in the FIG. 1A, following auditory fear conditioning, rats showed similar levels of freezing. On the following day, tones were paired with either VNS or sham stimulation, and 20 tones sounded alone in another group to facilitate extended extinction. Results showed that the VNS-treated group exhibited superior extinction to the sham-treated group and equivalent extinction to the group that were exposed to 5 times more tones during extinction training, indicating that VNS accelerates extinction.

A more recent study used a PTSD model, which involves presentation of a more stressful stimulus relative to that from auditory fear conditioning. Following 11 consecutive days of training, five of which were paired with either VNS or sham stimulation, only the VNS-treated group reached remission of fear, freezing on less than 10% of conditioned stimulus presentations (FIG. 1B). VNS-treated rats also showed no reinstatement of fear when tested 2 weeks later.

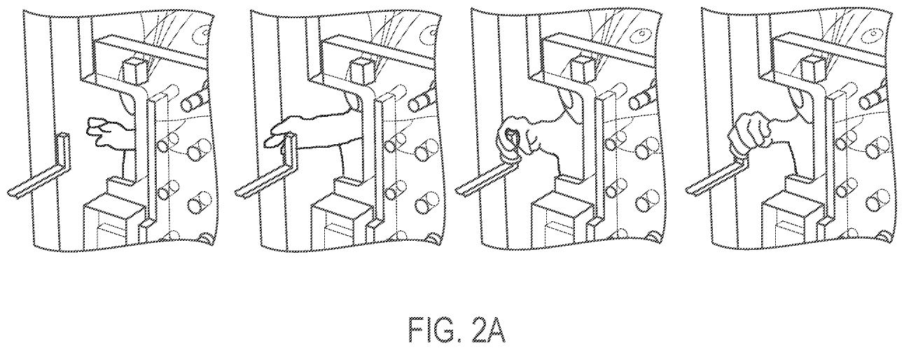

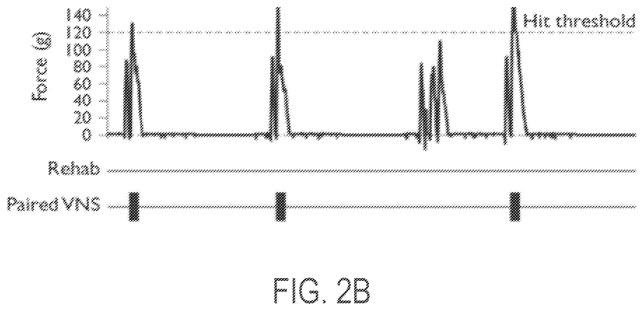

VNS is also being explored as an adjunct to treat movement impairments that result from neurological injury to the corticospinal system, such as with stroke or spinal cord injury. Similar to PTSD, the theoretical mechanism of action is increased neuromodulatory (e.g., noradrenergic) activity in the brain which enhances memory consolidation during motor re-learning. A paradigm used in several rodent studies involves training the rat to pull on a strain gauge before lesioning the corticospinal tract (FIG. 2). Re-emphasizing the importance of pairing VNS with behavioral or environmental events, these studies often involve a target force that the paretic limb must achieve in order for VNS to be administered. The objective is to reinforce patterns of cortical activation that produce proficient motor output from the paretic limb.

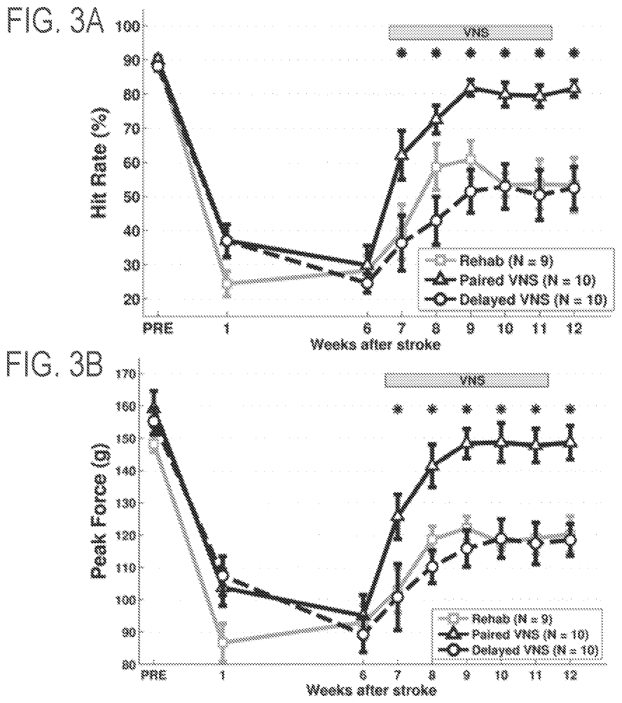

An accumulation of evidence from these animal studies demonstrates that pairing VNS with motor retraining leads to superior improvements in motor function when compared to motor retraining alone. Results of the study shown in FIG. 3 reinforce the importance of the temporal coincidence of pairing stimulation with behavior or environmental events, as another group that received delayed VNS does not exhibit the same degree of improved motor function.

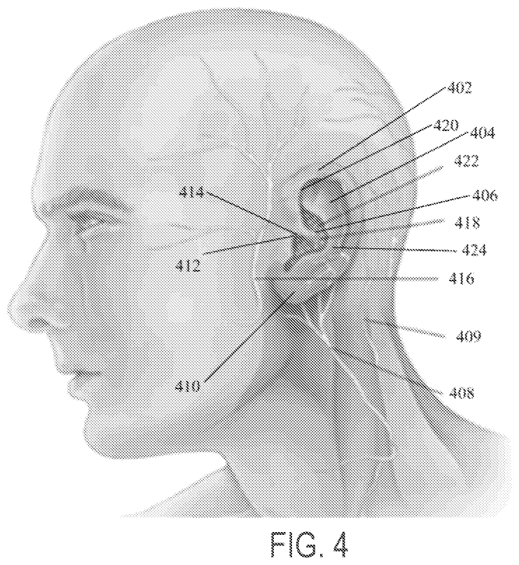

In light of preclinical findings, a number of implantable stimulators have been developed and are currently on the market. These devices have some drawbacks such as the need to undergo a surgical procedure that is both expensive and not reimbursed by most insurances. Such factors have led to the development of devices that seek to recruit the vagus nerve noninvasively through transcutaneous stimulation. A noninvasive approach to VNS appears plausible given human cadaver evidence of dense innervation in the ear from the auricular branch of the vagus nerve (FIG. 4). As shown in FIG. 4, the ear can have a helix 402, an antihelix 404, a concha 406, a great auricular nerve 408, a lesser occipital nerve 409, a lobule 410, an external auditory meatus 412, a tragus 414, an auriculotermporal nerve 416, and an auricular branch of the vagus nerve 418. The ear can have a first portion 420 associated with the auriculotemporal nerve, a second portion 422 associated with the auricular branch of the vagus nerve, and a third portion 424 associated with the great auricular nerve. A growing number of published studies have reported promising treatment effects, but there is no evidence that auricular stimulation activates one or more neuromodulatory mechanisms (e.g., optionally, the noradrenergic mechanisms) thought to underlie the therapeutic benefits of VNS. In fact, there are several published reports of null findings from auricular stimulation on changes in pupil diameter, which is an established biomarker of noradrenergic activation. This is problematic in that pairing activation of the noradrenergic system with behavioral and/or environmental events appears to be a critical determinant of treatment effects. Without verification that the system is engaged, it is impossible to ensure the incidence of nor the temporal coincidence of activation with external events.

### 2. Method

The one or more neuromodulatory systems (e.g., the noradrenergic system) can be activated via transcutaneous stimulation of the external ear. As further disclosed herein, a device can be developed to replicate electrode placement and stimulation parameters in a form factor for use in clinical and research settings. Pulse waveforms are generated by custom software and output by a commercially-available, isolated bipolar constant current stimulator (DS5, Digitimer Ltd, UK). Electrical current is controlled via custom software and a data acquisition device. Pulse trains consist of symmetric, biphasic waveforms with pre-specified amplitudes tailored to the individual subject. Pulse width, frequency and overall train duration is fixed.

To verify activation of the one or more neuromodulatory systems (e.g., the noradrenergic system), recordings of pupil dilation were obtained from 19 neurologically-intact adults. Modulation of pupil diameter is an established biomarker of the noradrenergic system. A three-part experimental procedure is carried out to 1) configure hardware/software and apply electrode interface, 2) establish perceptual threshold, and 3) to measure changes in pupil diameter resulting from stimulation.

First, the skin overlying the targeted landmark on the ear is lightly abraded and cleansed with preparation gel. Subsequently, the apparatus 10 can be inserted into the ear canal.

Next, the subject undergoes a 0.1 mA up/0.3 mA-down staircase procedure to establish perceptual thresholds, determining the minimum electrical current needed to evoke a percept. Perceptual threshold is taken as the average amplitude after eight reversals. The subject is instructed to raise his/her left hand when stimulation is perceived at the targeted location on the external ear. Two thresholding procedures are administered for reliability purposes, and the average of the two is taken as the perceptual threshold to calculate electrical current amplitudes entered into a custom program controlling the stimulating device during eye tracking procedures. Perceptual threshold is established for a given combination of stimulation parameters prior to eye tracking procedures.

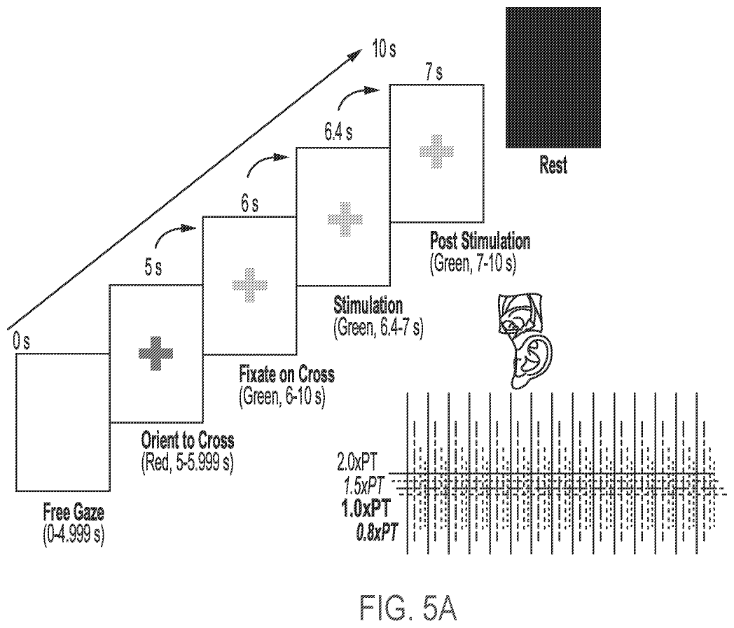

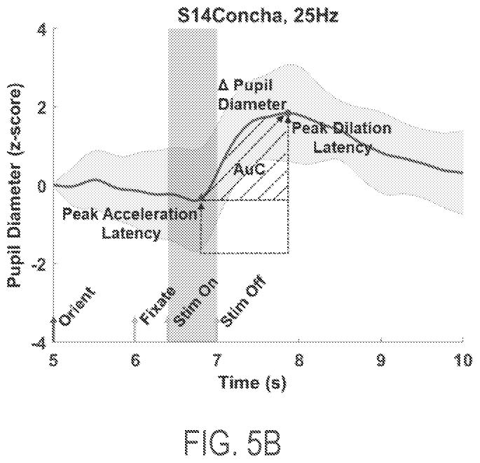

Then, saccades and pupil diameter are recorded (500 Hz sample rate) continuously while a series of visual cues configured in software native to a commercially available eye tracking system are presented on a computer monitor (FIG. 5A). Visual cues serve to provide instruction to the participant. The head and chin of the subject are positioned in a mounted frame, and room lights are turned off while measurements are obtained. Following a calibration procedure, the computer monitor background de-illuminates for 10 s to allow the participant an opportunity to close and/or relax their eyes. The background of the monitor illuminates immediately thereafter, at which time the participant minimizes blinks but is able to gaze freely. A fixation cross is presented on the monitor at 5 s, cueing the participant to orient gaze to the cross and avoid blinking entirely. The color of the cross changes from red to green at 6 s, signaling the participant to maintain fixation on the center of the cross until it disappears from the monitor at 10 s. Saccades alter pupil diameter so fixation is necessary to dissociate the effects of stimulation on pupil diameter. Pulse trains are administered 400 ms after the cross changes color from red to green (i.e., 6.4 s). To minimize transient effects of stimuli that are possible in pupillometry, a ˜9-second interval elapses between single stimulation trains. A series of consecutive trials are recorded before the monitor background de-illuminates again to allow the participant an opportunity to rest their eyes, resulting in one block of testing. A given combination of stimulation parameters is administered on an equal number of trials in random order within each testing block. A pre-specified number of testing blocks are administered before the participant is able to withdraw their head from the frame, resulting in a complete set of testing. This procedure is repeated to test the effects of stimulation to different locations and/or combinations of stimulation parameters at a given location. To minimize the potential for carryover effects, a 10-minute rest period is taken between sets.

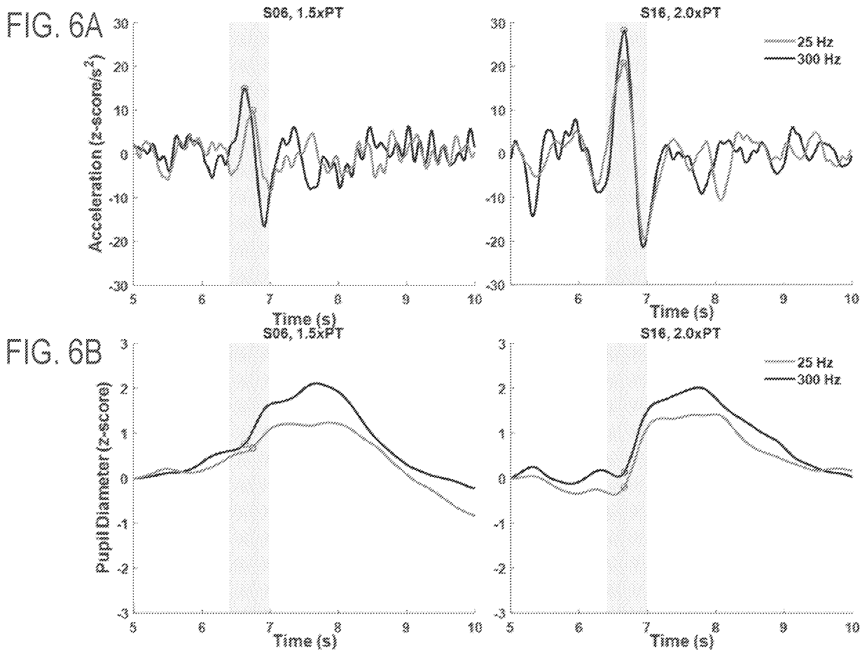

In accordance with standard data processing guidelines for pupillometry, all pupillary responses features are calculated from the waveform average of all pulse trains administered at a given location and fixed combination of stimulation parameters. FIG. 5B shows a representative waveform-averaged pupillary response depicting different features that represent the timing and size of the response. For response timing, the magnitude and latency of the peak positive acceleration within the stimulation epoch (6.4-7.05 s) is used to index response onset (FIG. 6A&B). Latency of peak dilation is measured between the time of peak acceleration and 1.6 s after stimulation onset (6.4-8 s). For response size, the change in pupil diameter between the time of peak acceleration and peak dilation is quantified. Since the time course to peak dilation can vary and be more or less sustained depending on how pulse trains influence postsynaptic firing in locus coeruleus, area under the curve (AuC) is quantified between the times of peak acceleration and peak dilation.

### 3. Results

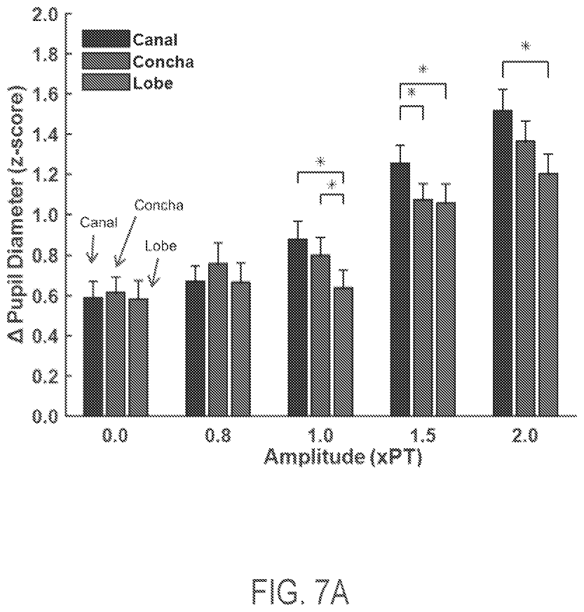

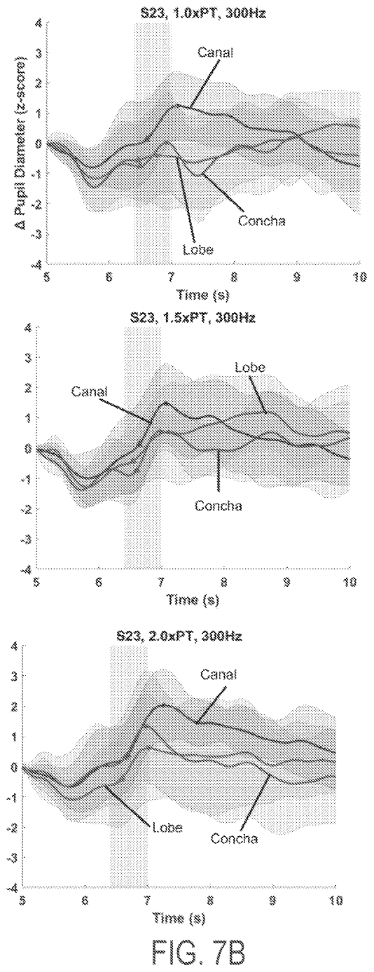

Shown in FIG. 7A is the change in pupil diameter in the sample of subjects resulting from pulse trains applied at each location on the external ear. A clear modulation is evident for pulse amplitudes at and above perceptual threshold, particularly for the canal relative to other locations. Shown in FIG. 7B are waveform averaged responses elicited by pulse trains applied to each location from a representative subject with pulse amplitudes at and above perceptual threshold. Note that activation of nociceptors mediating pain perception can engage an autonomic response that dilates the pupil. Determining whether the vagus nerve is recruited via a noninvasive approach in humans is not straightforward because the vagus nerve is composed of A, B, and C fiber types. To determine if nociceptors were involved in mediating responses, subjects were asked to report any instance of pain. Of the 19 subjects tested, one reported mild dizziness at the 2.0xPT amplitude. There were no reports of pain or discomfort.

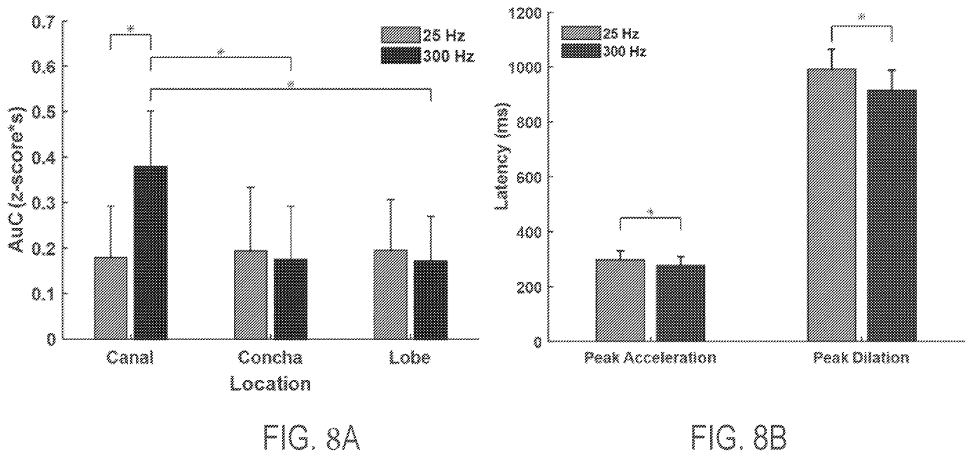

Shown in FIG. 8A&B are the effects of pulse frequency on metrics of pupillary response size and timing. AuC modulated with pulse frequency, but effects were contingent on location and pulse amplitude. Targeting the canal with higher pulse frequency, in particular, tended to elicit stronger pupillary responses (FIG. 8A). Higher pulse frequency reduced the latencies of peak acceleration and peak dilation of pupil diameter, thus, decreasing time elapsing between stimulation and the onset and peak of pupillary responses (FIG. 8B).

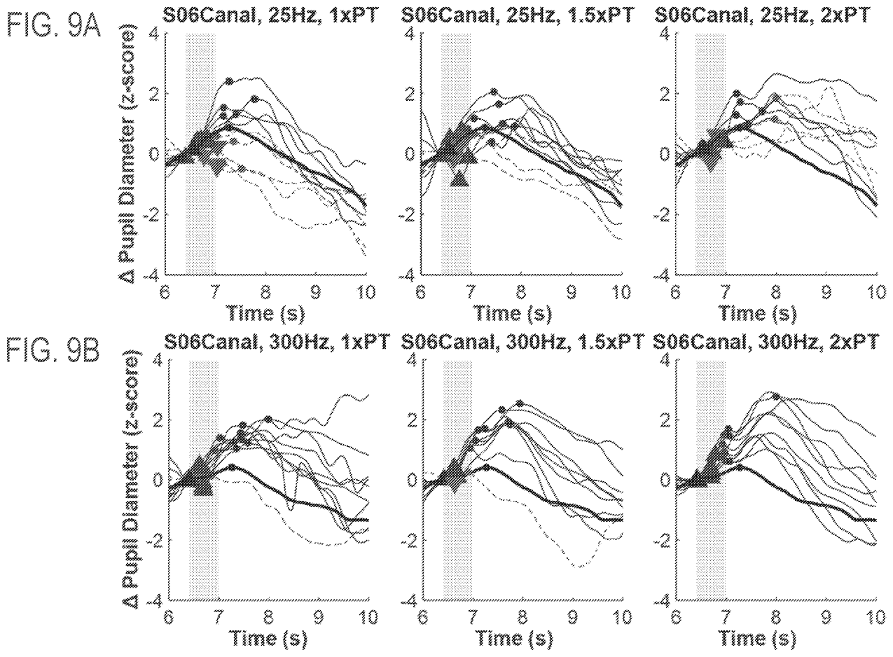

Inspection of pupil diameter recordings from individual pulse trains revealed instances with strong and abrupt modulations in pupil diameter shortly after the time of stimulation onset. A 0.0xPT pulse amplitude was randomized into each block of trials, serving as a reference of noradrenergic activity during eye tracking procedures, which is likely influenced by both tonic and phasic firing of locus coeruleus. Accepted standards for characterizing pupillary responses at the trial level have not been defined. The incidence of pupillary responses from individual pulse trains in the present study, therefore, was estimated by calculating the percentage of trials where the peak positive acceleration during the stimulation epoch and subsequent peak dilation nominally exceeded both features when the 0.0xPT pulse amplitude was applied. Based on these criteria, pupillary responses were elicited by single pulse trains. Shown in FIG. 9A&B are recordings of pupil diameter from single pulse trains at different pulse frequencies (rows) and amplitudes (columns) in a subject.

### 4. Conclusion

The findings indicate that the electrode interface described herein elicits autonomic responses in the form pupillary dilation. The magnitude and timing of effects in absence of pain percepts point to activation of one or more neuromodulatory systems, which are thought to contribute to the adaptive effects of both invasive and noninvasive VNS.

### Exemplary Aspects

In view of the described products, systems, and methods and variations thereof, herein below are described certain more particularly described aspects of the invention. These particularly recited aspects should not however be interpreted to have any limiting effect on any different claims containing different or more general teachings described herein, or that the “particular” aspects are somehow limited in some way other than the inherent meanings of the language literally used therein.
Aspect 1: An apparatus for use with a user having an ear, the ear having an ear canal, a tragus, and a concha, the apparatus comprising:
a body that is at least partly receivable into the ear canal, the body having an outer surface; and
a plurality of electrodes that are coupled to the body, wherein the plurality of electrodes are positioned to contact surfaces of the ear.
Aspect 2: The apparatus of aspect 1, wherein the body comprises biocompatible elastomer.
Aspect 3: The apparatus of aspect 1, wherein the body comprises a first portion that is receivable into the ear canal of the user and a second portion that is configured to extend outwardly from the ear canal.
Aspect 4: The apparatus of aspect 3, wherein the outer surface is complementary to a shape of the ear canal of the user based on a scan or mold of the ear canal.
Aspect 5: The apparatus of aspect 3 or aspect 4, wherein the second portion of the body comprises a protrusion that is configured to extend over the tragus of the ear, wherein the plurality of electrodes comprises at least one electrode coupled to the protrusion of the second portion of the body.
Aspect 6: The apparatus of any one of aspects 3-5, wherein the second portion of the body is configured to contact surfaces of the concha of the ear.
Aspect 7: The apparatus of any one of the preceding aspects, wherein the plurality of electrodes comprise adjacent electrodes that are spaced apart by a spacing from about 0.2 mm to about 5.0 mm.
Aspect 8: The apparatus of any one of aspects 3-7, wherein the first portion of the body is elongated along a first axis, wherein the plurality of electrodes comprises a first plurality of electrodes circumferentially spaced about the first axis.
Aspect 9: The apparatus of aspect 8, wherein the first plurality of electrodes circumferentially spaced about the first axis comprise pairs of electrodes that are spaced along the first axis.
Aspect 10: The apparatus of aspect 8, wherein the first plurality of electrodes circumferentially spaced about the first axis comprise single electrodes that are circumferentially spaced about the first portion of the body.
Aspect 11: The apparatus of any one of the preceding aspects, wherein the plurality of electrodes are coupled to the outer surface of the body.
Aspect 12: The apparatus of any one of the preceding aspects, wherein the plurality of electrodes are at least partly recessed within the body.
Aspect 13: The apparatus of any one of the preceding aspects, wherein the plurality of electrodes extend outwardly from the outer surface of the body.
Aspect 14: The apparatus of any one of aspects 3-9, wherein the second portion of the body is configured to extend outwardly from the ear canal by at least 4 mm when the first portion of the body is fully received within the ear canal.
Aspect 15: The apparatus of any one of the preceding aspects, wherein the body is flexible.
Aspect 16: The apparatus of any one of the preceding aspects, further comprising a plurality of electrical leads that couple to and extend from respective electrodes of the plurality of electrodes.
Aspect 17: The apparatus of any one of the preceding aspects, wherein the body defines at least one opening that is configured to communicate sound therethrough.
Aspect 18: The apparatus of aspect 17, wherein the body comprises open cell foam.
Aspect 19: The apparatus of aspect 17, wherein the body defines a conduit that extends therethrough for communicating sound through the body.
Aspect 20: The apparatus of aspect 19, further comprising a plurality of electrical leads that couple to and extend from respective electrodes of the plurality of electrodes, wherein the plurality of electrodes extend through the conduit.
Aspect 21: The apparatus of any one of the preceding aspects, wherein at least one electrode of the plurality of electrodes comprises:
a housing comprising:
   a first housing portion defining a skin contact surface of the electrode; and
   second housing portion that cooperates with the first housing portion to define an enclosure, wherein the second housing portion of the housing defines an opening; and
a conductor extending through the opening, wherein the conductor has a first end that is soldered to the first housing portion and second housing portion so that solder is contained within the enclosure of the housing.
Aspect 22: A method of directly or secondarily activating one or more neuromodulatory systems in a subject comprising:
applying one or more electrical pulses to an ear of the subject,
wherein the one or more electrical pulses are sufficient to directly or secondarily activate the one or more neuromodulatory systems of the subject,
wherein the one or more electrical pulses are applied using an apparatus as in any one of aspects 1-21.
Aspect 23: The method of aspect 22, wherein applying one or more electrical pulses to the ear of the subject comprises:
positioning at least a portion of a device on an outer ear of the ear of the subject; and
generating electrical pulses with the device, wherein the electrical pulses are transmitted through the ear to a nerve in the subject.
Aspect 24: The method of aspect 22, wherein the one or more electrical pulses are applied unilaterally to the subject.
Aspect 25: The method of aspect 24, wherein the one or more electrical pulses are applied to one of a left ear or a right ear of the subject.
Aspect 26: The method of aspect 22, wherein the one or more electrical pulses are applied bilaterally to the subject.
Aspect 27: The method of aspect 22, wherein directly or secondarily activating the one or more neuromodulatory systems occurs via stimulation to the nerve.
Aspect 28: The method of aspect 22, wherein the nerve is a vagus nerve having an auricular branch, and wherein the electrical pulses are transmitted through the ear to the auricular branch of the vagus nerve.
Aspect 29: The method of aspect 22, wherein positioning a device on the ear of the subject comprises inserting a portion of the body of the apparatus into an ear canal.
Aspect 30: The method of aspect 22, wherein activation of the one or more neuromodulatory systems is confirmed by assessing pupil dilation.
Aspect 31: The method of aspect 30, wherein assessing pupil dilation comprises:
quantifying baseline pupil dilation; establishing perceptual thresholds; and
measuring changes in pupil dilation from baseline after activation of the one or more neuromodulatory systems.
Aspect 32: The method of aspect 22, wherein the one or more electrical pulses are administered at a frequency of between 1 Hz and 10,000 Hz.
Aspect 33: The method of aspect 22, wherein applying one or more electrical pulses to an ear canal of the subject occurs for a duration of about 1 ms to 30 minutes.
Aspect 34: The method of aspect 22, wherein applying one or more electrical pulses to the ear of the subject comprises applying one or more electrical pulses to the left external ear of the subject.
Aspect 35: The method of aspect 34, wherein applying one or more electrical pulses to the left external ear of the subject comprises applying one or more electrical pulses to the left ear canal of the subject.
Aspect 36: A method of treating a disease or disorder in a subject comprising:
applying one or more electrical pulses to an ear of the subject, wherein the one or more electrical pulses are sufficient to directly or secondarily activate the one or more neuromodulatory systems of the subject such that the disease or disorder in the subject is treated,
wherein the one or more electrical pulses are applied using an apparatus as in any one of aspects 1-21.
Aspect 37: The method of aspect 36, wherein the one or more electrical pulses are applied unilaterally to a left ear or a right ear of the subject.
Aspect 38: The method of aspect 37, wherein the one or more electrical pulses are applied bilaterally to the subject.
Aspect 39: The method aspect 36, wherein applying one or more electrical pulses to a nerve of the subject comprises applying the one or more electrical pulses according to a treatment paradigm.
Aspect 40: The method of aspect 39, wherein the treatment paradigm comprises a train of electrical pulses for a time period of about 1 ms to about 30 minutes as a single dose.
Aspect 41: The method of aspect 39, wherein the treatment paradigm is applied once daily.
Aspect 42: The method of aspect 39, wherein the treatment paradigm is applied at least twice daily.
Aspect 43: The method of aspect 42, wherein each treatment paradigm is applied within 5 minutes of a preceding treatment paradigm.
Aspect 44: The method of aspect 39, wherein the treatment paradigm is applied once a week.
Aspect 45: The method of aspect 39, wherein the treatment paradigm is applied once a month.
Aspect 46: The method of aspect 39, wherein the disease or disorder is a psychiatric disorder, neurologic disorder, a chronic inflammatory disorder, substance abuse, or addiction.
Aspect 47: The method of aspect 46, wherein the psychiatric disorder is post-traumatic stress disorder (PTSD), anxiety, depression, schizophrenia, or motor impairments resulting from neurological injury to the corticospinal tract.
Aspect 48: The method of aspect 46, wherein the neurological disorder is epilepsy or paresis.
Aspect 49: The method of aspect 46, wherein the chronic inflammatory disorder is fibromyalgia, migraine headaches, or obesity.
Aspect 50: The method of aspect 36, wherein direct or secondary activation of the one or more neuromodulatory systems extinguishes conditioned fears through repeated reminders of traumatic events.
Aspect 51: The method of aspect 36, wherein direct or secondary activation of the one or more neuromodulatory systems improves consolidation and maintenance of the extinction memory.
Aspect 52: The method of aspect 36, wherein directly or secondarily activating the one or more neuromodulatory systems comprises stimulating the nerve of the subject.
Aspect 53: A method of ameliorating a symptom associated with a psychiatric disorder, neurological disorder, or chronic inflammatory disorder in a subject comprising applying one or more electrical pulses to a left ear canal of the subject with the apparatus as in any one of aspects 1-21, wherein the one or more electrical pulses are sufficient to ameliorate a symptom associated with a psychiatric disorder, neurological disorder, or chronic inflammatory disorder in a subject.
Aspect 54: A system comprising:
an apparatus as in any one of aspects 1-21; and
a stimulation generator in communication with the first electrical conductor and the second electrical conductor, wherein the stimulation generator is configured to generate current for providing electrical stimulation through or between the first and second electrodes of the apparatus.
Aspect 55: The system of aspect 54, further comprising:
at least one processor; and
memory in communication with the at least one processor, wherein the memory comprises instructions that, when executed by the at least one processor, cause the at least one processor to:
   receive data from the apparatus.
Aspect 56: The system of aspect 55, further comprising:
an input device in communication with the at least one processor; and
a display device,
wherein the memory comprises instructions that, when executed by the at least one processor, cause the at least one processor to:
   cause the display device to display the data received from the apparatus; and
   receive an input from a clinician to enable the clinician to adjust at least one parameter of the stimulation generator based on the received data.
Aspect 57: The system of aspect 56, wherein the memory comprises instructions that, when executed by the at least one processor, cause the at least one processor to automatically adjust at least one parameter of the stimulation generator based on the received data.
Aspect 58: The system of aspect 57, wherein the at least one parameter comprises a stimulation amplitude or a stimulation frequency.
Aspect 59: A kit comprising:
a plurality of apparatuses as in any one of aspects 1-21, wherein each apparatus of the plurality of apparatuses differs from the other of the plurality of apparatuses by at least one of: a spacing between the first electrode and the second electrode, a dimension of the first electrode, a dimension of the second electrode, a shape of the first electrode, or a shape of the second electrode.
Aspect 60: A method of directly or secondarily activating the one or more neuromodulatory systems in a subject comprising applying one or more electrical pulses to an ear of the subject using an apparatus as in any one of aspects 1-21,
wherein the one or more electrical pulses are sufficient to directly or secondarily activate the one or more neuromodulatory systems of the subject.
Aspect 61: The method of aspect 60, wherein applying one or more electrical pulses to the ear of the subject comprises:
inserting a portion of the body of the apparatus into an ear canal of the ear of the subject; and
generating electrical pulses with the device, wherein the electrical pulses are transmitted through the ear to a nerve in the subject.
Aspect 62: The method of aspect 60, wherein the one or more electrical pulses are applied unilaterally to the subject.
Aspect 63: The method of aspect 62, wherein the one or more electrical pulses are applied to one of a left ear or a right ear of the subject.
Aspect 64: The method of aspect 60, wherein the one or more electrical pulses are applied bilaterally to the subject.
Aspect 65: The method of aspect 60, wherein directly or secondarily activating the one or more neuromodulatory systems occurs via stimulation to the nerve.
Aspect 66: The method of aspect 60, wherein the nerve is a vagus nerve having an auricular branch, and wherein the electrical pulses are transmitted through the ear to the auricular branch of the vagus nerve.
Aspect 67: The method of aspect 60, wherein activation of the one or more neuromodulatory systems is confirmed by assessing pupil dilation.
Aspect 68: The method of aspect 67, wherein assessing pupil dilation comprises:
quantifying baseline pupil dilation;
establishing perceptual thresholds; and
measuring changes in pupil dilation from baseline after activation of the one or more neuromodulatory systems.
Aspect 69: The method of aspect 60, wherein the one or more electrical pulses are administered at a frequency of between 1 Hz and 10,000 Hz.
Aspect 70: The method of aspect 60, wherein applying one or more electrical pulses to an car canal of the subject occurs for a duration of about 1 ms to 1000 ms.
Aspect 71: The method of aspect 60, wherein applying one or more electrical pulses to an ear of the subject comprises applying one or more electrical pulses to the left external ear of the subject.
Aspect 72: The method of aspect 71, wherein applying one or more electrical pulses to the left external ear of the subject comprises applying one or more electrical pulses to the left ear canal of the subject.
Aspect 73: A method of treating a disease or disorder in a subject comprising:
applying one or more electrical pulses to at least one of a left ear or a right ear of a subject using an apparatus as in any one of aspects 1-21, wherein the one or more electrical pulses are sufficient to directly or secondarily activate the one or more neuromodulatory systems of the subject such that the disease or disorder in the subject is treated.
Aspect 74: The method of aspect 73, wherein the one or more electrical pulses are applied unilaterally to one ear of the subject.
Aspect 75: The method of aspect 74, wherein the one or more electrical pulses are applied simultaneously to both ears of the subject.
Aspect 76: The method of aspect 73, wherein applying one or more electrical pulses to a nerve of the subject comprises applying the one or more electrical pulses according to a treatment paradigm.
Aspect 77: The method of aspect 76, wherein the treatment paradigm comprises a train of electrical pulses for a time period of about 1 ms to about 30 minutes as a single dose.
Aspect 78: The method of aspect 76, wherein the treatment paradigm is applied once daily.
Aspect 79: The method of aspect 76, wherein the treatment paradigm is applied at least twice daily.
Aspect 80: The method of aspect 79, wherein each treatment paradigm is applied within 5 minutes of a preceding treatment paradigm.
Aspect 81: The method of aspect 76, wherein the treatment paradigm is applied once a week.
Aspect 82: The method of aspect 76, wherein the treatment paradigm is applied once a month.
Aspect 83: The method of aspect 73, wherein the disease or disorder is a psychiatric disorder, neurologic disorder, a chronic inflammatory disorder, substance abuse, or addiction.
Aspect 84: The method of aspect 83, wherein the psychiatric disorder is post-traumatic stress disorder (PTSD), anxiety, depression, schizophrenia, or motor impairments resulting from neurological injury to the corticospinal tract.
Aspect 85: The method of aspect 83, wherein the neurological disorder is epilepsy or paresis.
Aspect 86: The method of aspect 83, wherein the chronic inflammatory disorder is fibromyalgia, migraine headaches, or obesity.
Aspect 87: The method of aspect 73, wherein direct or secondary activation of the one or more neuromodulatory systems extinguishes conditioned fears through repeated reminders of traumatic events.
Aspect 88: The method of aspect 73, wherein direct or secondary activation of the one or more neuromodulatory systems improves consolidation and maintenance of the extinction memory.
Aspect 89: The method of aspect 73, wherein directly or secondarily activating the noradrenergic system comprises stimulating the nerve of the subject.

Those skilled in the art will recognize, or be able to ascertain using no more than routine experimentation, many equivalents to the specific embodiments of the method and compositions described herein. Such equivalents are intended to be encompassed by the following claims.

1. An apparatus for use with a user having an ear, the ear having an ear canal, a tragus, and a concha, the apparatus comprising:
a body that is at least partly receivable into the ear canal, the body having an outer surface; and
a plurality of electrodes that are coupled to the body, wherein the plurality of electrodes are positioned to contact surfaces of the ear.

2. (canceled)

3. The apparatus of claim 1, wherein the body comprises a first portion that is receivable into the ear canal of the user and a second portion that is configured to extend outwardly from the ear canal.

4. The apparatus of claim 3, wherein the outer surface of the body is complementary to a shape of the ear canal of the user based on a scan or mold of the ear canal, and wherein the second portion of the body comprises a protrusion that is configured to extend over the tragus of the ear, wherein the plurality of electrodes comprises at least one electrode coupled to the protrusion of the second portion of the body.

5. (canceled)

6. The apparatus of claim 3, wherein the second portion of the body is configured to contact surfaces of the concha of the ear.

7. The apparatus of claim 1, wherein the plurality of electrodes comprise adjacent electrodes that are spaced apart by a spacing from about 0.2 mm to about 5.0 mm.

8. The apparatus of claim 3, wherein the first portion of the body is elongated along a first axis, wherein the plurality of electrodes comprises a first plurality of electrodes circumferentially spaced about the first axis, and wherein the first plurality of electrodes circumferentially spaced about the first axis comprise pairs of electrodes that are spaced along the first axis.

9. (canceled)

10. The apparatus of claim 3, wherein the first portion of the body is elongated along a first axis, wherein the plurality of electrodes comprises a first plurality of electrodes circumferentially spaced about the first axis, and wherein the first plurality of electrodes circumferentially spaced about the first axis comprise single electrodes that are circumferentially spaced about the first portion of the body.

11. (canceled)

12. The apparatus of claim 1, wherein the plurality of electrodes are at least partly recessed within the body.

13. The apparatus of claim 1, wherein the plurality of electrodes extend outwardly from the outer surface of the body.

14. The apparatus of claim 3, wherein the second portion of the body is configured to extend outwardly from the ear canal by at least 4 mm when the first portion of the body is fully received within the ear canal.

15. (canceled)

16. (canceled)

17. The apparatus of claim 1, wherein the body defines at least one opening that is configured to communicate sound therethrough.

18. The apparatus of claim 17, wherein the body comprises open cell foam.

19. (canceled)

20. (canceled)

21. The apparatus of claim 1, wherein at least one electrode of the plurality of electrodes comprises:
a housing comprising:
a first housing portion defining a skin contact surface of the electrode; and
a second housing portion that cooperates with the first housing portion to define an enclosure, wherein the second housing portion of the housing defines an opening; and
a conductor extending through the opening, wherein the conductor has a first end that is soldered to the first housing portion and second housing portion so that solder is contained within the enclosure of the housing.

22. A method of directly or secondarily activating one or more neuromodulatory systems in a subject comprising:
applying one or more electrical pulses to an ear of the subject,
wherein the one or more electrical pulses are sufficient to directly or secondarily activate the one or more neuromodulatory systems of the subject,
wherein the one or more electrical pulses are applied using an apparatus as in claim 1.

23-35. (canceled)

36. A method of treating a disease or disorder in a subject comprising:
applying one or more electrical pulses to an ear of the subject, wherein the one or more electrical pulses are sufficient to directly or secondarily activate the one or more neuromodulatory systems of the subject such that the disease or disorder in the subject is treated,
wherein the one or more electrical pulses are applied using an apparatus as in claim 1.

37-52. (canceled)

53. A method of ameliorating a symptom associated with a psychiatric disorder, neurological disorder, or chronic inflammatory disorder in a subject comprising applying one or more electrical pulses to a left ear canal of the subject with the apparatus as in claim 1, wherein the one or more electrical pulses are sufficient to ameliorate a symptom associated with a psychiatric disorder, neurological disorder, or chronic inflammatory disorder in a subject.

54. A system comprising:
an apparatus as in claim 1; and
a stimulation generator in communication with the first electrical conductor and the second electrical conductor, wherein the stimulation generator is configured to generate current for providing electrical stimulation through or between the first and second electrodes of the apparatus.

55-58. (canceled)

59. A kit comprising:
a plurality of apparatuses as in claim 1, wherein each apparatus of the plurality of apparatuses differs from the other of the plurality of apparatuses by at least one of: a spacing between the first electrode and the second electrode, a dimension of the first electrode, a dimension of the second electrode, a shape of the first electrode, or a shape of the second electrode.

60. A method of directly or secondarily activating one or more neuromodulatory systems in a subject comprising applying one or more electrical pulses to an ear of the subject using an apparatus as in claim 1,
wherein the one or more electrical pulses are sufficient to directly or secondarily activate the one or more neuromodulatory systems of the subject.

61-72. (canceled)

73. A method of treating a disease or disorder in a subject comprising:
applying one or more electrical pulses to at least one of a left ear or a right ear of a subject using an apparatus as in claim 1, wherein the one or more electrical pulses are sufficient to directly or secondarily activate the one or more neuromodulatory systems of the subject such that the disease or disorder in the subject is treated.

74-89. (canceled)
